(19) United States
(12) Patent Application Publication
(10) Pub. No.: US 2024/0277896 A1
(43) Pub. Date: Aug. 22, 2024

(54) CATECHOLAMINE-BASED MEMBRANE, PROCESS FOR ITS PREPARATION AND USES THEREOF

(71) Applicants: FUNDACIÓ INSTITUT CATALÀ DE NANOCIÈNCIA I NANOTECNOLOGIA (ICN2), CERDANYOLA DEL VALLÈS (ES); CONSEJO SUPERIOR DE INVESTIGACIONES CIENTÍFICAS (CSIC), MADRID (ES)

(72) Inventors: Salvio SUÁREZ GARCÍA, CORNELLÀ DE LLOBREGAT (ES); Javier SAIZ POSEU, MANRESA (ES); Daniel RUIZ MOLINA, SABADELL (ES)

(73) Assignees: FUNDACIÓ INSTITUT CATALÀ DE NANOCIÈNCIA I NANOTECNOLOGIA (ICN2), CERDANYOLA DEL VALLÈS (ES); CONSEJO SUPERIOR DE INVESTIGACIONES CIENTÍFICAS (CSIC), MADRID (ES)

(21) Appl. No.: 18/568,815

(22) PCT Filed: Jun. 9, 2022
(86) PCT No.: PCT/EP2022/065757

§ 371 Date: Dec. 9, 2023

(30) Foreign Application Priority Data
Jun. 10, 2021 (EP) 21382516.9

(51) Int. Cl. A61L 27/34; A61L 24/00; A61L 24/04; A61L 27/54; A61L 27/58; C08G 65/40; C08G 65/48; C08G 73/02

(52) U.S. Cl. CPC A61L 27/34; A61L 24/0015; A61L 24/0042; A61L 24/046; A61L 27/54; A61L 27/58; C08G 65/4075; C08G 65/48; C08G 73/0206; C08G 73/0213; C08G 73/0253; C08G 2150/00; C08G 2170/80; C08G 2230/00

(57) ABSTRACT

The present invention provides a process for preparing a self-standing catecholamine-based membrane, the process comprising the steps of: (a) cross-linking a catechol derivative with an amine selected from the group consisting of: a known aliphatic amine hydrocarbon of formula (II); and an aromatic amine of formula (IIbis), in a liquid medium, wherein both the catechol and the amine are soluble, at a pH comprised from 6.5 to 10, and under appropriate agitation to create a catecholamine membrane in the air/liquid interface in the absence of any support; and b) isolating the membrane resulting from step (a) from the air/liquid interface. The resulting self-standing catecholamine-based membrane was robust, easy to handle and manipulate, highly flexible and adaptable to any kind of surface without breaking, and adhesive. In addition, the free-standing membrane of the invention shows a Janus character, with an unexpected nanopatterning in the water-contact side which endows the membranes of the invention with a higher roughness surface, something of value to promote cell adhesion.

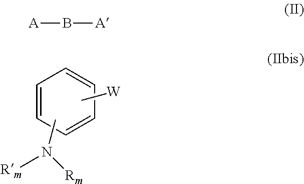

This application claims the benefit of the European Patent Application 21382516.9 filed on 10 Jun. 2021.

### TECHNICAL FIELD

The present invention relates to the field of catecholamine-based membranes. In particular, the present invention provides a self-standing catecholamine membrane, a process for its preparation and uses thereof.

### BACKGROUND ART

Catechol derivatives are widely distributed among natural, animal and plant systems, all of them characterized by sharing an aromatic core of formula (I):

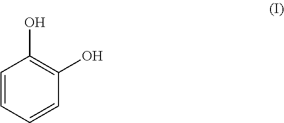

One famous example of a catechol derivative is 3,4-dihydroxyphenylalanine (DOPA). When exposed to air, DOPA is prone to oxidation. The formed o-quinones may further react with a variety of nucleophiles in various pathways to form crosslinks. A well-known nucleophile is the amine that may react with o-quinones to form adducts either by Michael addition or Schiff base reaction.

The reaction between catechol derivatives and amines is of vital importance in natural biological processes, such as the cross-linking of adhesive proteins by marine organisms, the formation of the cytoskeleton by insects and the biosynthesis of melanin. This catechol-amine chemistry occurring in natural organisms has attracted much attention in material science due to the increasing interest in finding new materials.

Thus, several research groups have developed water-soluble polymers that form a gel or water-resistant films upon reaction between a catechol derivative and an amine. Many publications have reported the preparation of catecholamine-based coatings. For example, lacomino M. and colleagues (lacomino M. et al., 2017) report the coating of a substrate by dip-coating with a solution comprising caffeic acid and a diamine crosslinker at a pH 9. The coating substrate was efficient in promoting cell growth. Suarez-Garcia S. and colleagues (Suarez-Garcia S. et al., 2017) have also reported the functionalization of substrate surfaces (such as glass, gold and silica) with thin films obtained by copolymerization of a catechol and a diamine.

However, these coated substrates, due to the rigidity conferred by the own substrate, limit their use. And the catecholamine coating is strongly adhered to the substrate, so it is not possible to detach it from the substrate without damaging it because the coating is not robust enough to stand the required detaching forces.

Due to the limitations shown by these coatings, in recent years, free-standing polymeric films with well-defined structural and adaptive functions have attracted interest. Various methods have been developed to prepare self-supporting polymer films, such as layer-by-layer self-assembly (LbL), Langmuir-Blodgett deposition methods, spin-coating and casting, and the like.

Accordingly, the research of polymer film is still strictly limited due to the lack of a convenient and efficient synthesis method at present. For example, self-supporting polymer films can be prepared from crosslinked, self-assembled monolayer films by surface-initiated polymerization reactions, which have very thin properties, as well as good chemical stability and sensitivity. However, this method requires the use of electron beams for crosslinking, and severe conditions may affect the broad application of the method. In order to make the conditions milder, a method including gas/liquid interface assembly or asymmetric modification was also studied, forming a polydopamine film at an air-water interface. However, the film material was easy to generate cracks during the preparation process, and the stability of the film was found to be poor. Other research groups have been studying the synthesis using polyethyleneimine (PEI) as a supporting material. For example, Ponzio F. and colleagues (Ponzio F. et al., 2016) disclosed the use of PEI to confer hardiness to a membrane-based on dopamine. The reaction conditions require a basic pH and under biological environment, the presence of PEI provides toxic by-products, limiting the use of these membranes.

In view of the above, there is still the need to provide robust, adaptable and safe catecholamine-based membranes as well as processes for their preparation.

### SUMMARY OF INVENTION

The present inventors have developed a new process for obtaining free-standing catecholamine-based membranes with very advantageous mechanical features, but also with a profile of degradability, adhesion and cell proliferation that make them especially useful in therapeutic and diagnostic applications.

As shown below, the process of the invention is based on the particular selection of an amine, either an aliphatic amine which is a hydrocarbon chain having a terminal amine at each end of the chain or an aromatic amine, as a crosslinker agent, as well as on the strict reaction conditions of pH and agitation. Remarkably, the pH reaction conditions are so mild that they allow the in situ functionalization with biological molecules, something advantageous when compared with processes of the prior art, which work under basic conditions.

Thus, in a first aspect, the invention provides a process for preparing a catecholamine-based membrane, the process comprising the steps of:
a) crosslinking a catechol derivative with an amine selected from the group consisting of:
a.1) a known aliphatic amine of formula (II)

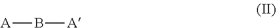

wherein
A and A′ are the same or different and represent —NR₁R′₁,
B represents a (C₁-C₂₀)alkylene; (C₁-C₂₀)alkylene substituted with one or more substituents selected from the group consisting of: —OH, halogen, —NO₂, cyano, —O—(C₁-C₁₀)alkyl, —C(O)OR₂, and —NR₃R′₃; (C₂-C₂₀)alkenylene; (C₂-C₂₀)alkenylene substituted with one or more substituents selected from the group consisting of: —OH, halogen, —NO₂, cyano, —O—(C₁-C₁₀)alkyl, —C(O)OR₄, and —NR₅R′₅; (C₂-C₂₀)alkynylene; (C₂-C₂₀)alkynylene substituted with one or more substituents selected from the group consisting of: —OH, halogen, —NO₂, cyano, —O—(C₁-C₁₀)alkyl, —C(O)OR₆, and —NR₇R′₇; a known 3- to 20-membered heteroalkylene; a known 3- to 20-membered heteroalkylene substituted with one or more substituents selected from the group consisting of: —OH, halogen, —NO₂, cyano, —O—(C₁-C₁₀)alkyl, —C(O)OR₈, and NR₉R′₉; a known 3- to 20-membered heteroalkenylene; and a known 3- to 20-membered heteroalkenylene substituted with one or more substituents selected from the group consisting of: —OH, halogen, —NO₂, cyano, —O—(C₁-C₁₀)alkyl, —C(O)OR₁₀, and —NR₁₁R′₁₁;
A is bound to the first atom member forming part of the B biradical hydrocarbon backbone;
A′ is bound to the last atom member forming part of the B biradical hydrocarbon backbone;
wherein the “first” and “last” atom members are identified reading the B biradical hydrocarbon backbone from left to right or vice versa;
R₁, R′₁, R₃, R′₃, R₅, R′₅, R₇, R′₇, R₉, R′₉, R₁₁, and R′₁₁ are the same or different and are selected from the group consisting of: —H; (C₁-C₁₀)alkyl; (C₁-C₁₀)haloalkyl; (C₂-C₁₀)alkenyl; (C₂-C₁₀)alkynyl; (C₁-C₁₀)alkyl substituted with one or more substituents selected from the group consisting of —OH, —NO₂, cyano, —O—(C₁-C₁₀)alkyl, —C(O)OR₁₂, and —NR₁₃R′₁₃;
R₂, R₄, R₆, R₈, R₁₀ and R₁₂ are independently selected from the group consisting of —H; (C₁-C₁₀)alkyl; (C₁-C₁₀)haloalkyl; (C₂-C₁₀)alkenyl; (C₂-C₁₀)alkynyl; (C₁-C₁₀)alkyl substituted with one or more substituents selected from the group consisting of —OH, —NO₂, cyano, —O—(C₁-C₁₀)alkyl, —C(O)OR₁₄ and —NR₁₅R′₁₅;
R₁₃, R′₁₃, R₁₄, R₁₅ and R′₁₅ are the same or different and are selected from the group consisting of: —H; (C₁-C₁₀)alkyl; (C₁-C₁₀)haloalkyl; (C₂-C₁₀)alkenyl; and (C₂-C₁₀)alkynyl;
a “known 3- to 20-membered heteroalkylene” means a known saturated chain consisting of from 3 to 20 members selected from the group consisting of C(Rₓ)₂, CRₓ, —N—, —NR′ₓ—, —S—, and —O—, provided that: (a) at least one of the members is —N—, —NRₓ—, —S—, or —O—; and (b) the first and the last members forming the backbone of the heteroalkylene are carbon atoms;
a “known 3 to 20-membered heteroalkenylene” means a known unsaturated chain consisting of from 3 to 20 members selected from the group consisting of C(Rₓ)₂, —CRₓ—, —N—, —NR′ₓ, —S—, and —O—, provided that: (a) at least one of the members is —N—, —NRₓ—, —S—, or —O—; (b) the first and the last members forming the backbone of the heteroalkylene are carbon atoms; and (c) includes one or more double bonds;
Rₓ is independently selected from the group consisting of —H; —OH; (C₁-C₁₀)alkyl; (C₂-C₁₀)alkenyl; (C₂-C₁₀)alkynyl; (C₁-C₁₀)haloalkyl; O—(C₁-C₁₀)alkyl; —O—(C₂-C₁₀)alkenyl; —O—(C₂-C₁₀)alkynyl; nitro, —NRₓ₁Rₓ₂; (C₁-C₁₀)alkyl substituted with one or more substituents selected from the group consisting of —OH, —NO₂, cyano, —O—(C₁-C₁₀)alkyl, —C(O)OR₁₆, and —NR₁₇R′₁₇; and halogen; and
R_{X1}, Rₓ₂, R₁₆, R₁₇, R′₁₇, and R′ₓ are independently selected from the group consisting of —H, (C₁-C₁₀)alkyl, (C₂-C₁₀)alkenyl, (C₂-C₁₀)alkynyl, and (C₁-C₁₀)haloalkyl; and
a.2) an aromatic amine of formula (IIbis):

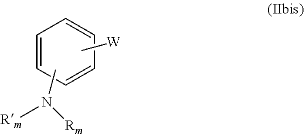

wherein W represents:
—NRₜR′ₜ or
*S-S-L; wherein (*) denotes that the S atom of W radical is bound to a carbon atom forming part of the aromatic ring; and
   L is selected from the group consisting of: (C₁-C₁₀)alkyl substituted with one or more substituents selected from the group consisting of —OH, —NO₂, cyano, —O—(C₁-C₁₀)alkyl, —C(O)OR₂₈, and —NR₂₈R′₂₈, provided that at least one of the substituents is —NR₂₈R′₂₈; (C₁-C₁₀)haloalkyl substituted with one or more substituents selected from the group consisting of —OH, —NO₂, cyano, —O—(C₁-C₁₀)alkyl, —C(O)OR₂₈, and —NR₂₈R′₂₈, provided that at least one of the substituents is —NR₂₈R′₂₈; (C₂-C₁₀)alkenyl substituted with one or more substituents selected from the group consisting of —OH, —NO₂, cyano, —O—(C₁-C₁₀)alkyl, —C(O)OR₂₈, and —NR₂₈R′₂₈, provided that at least one of the substituents is —NR₂₈R′₂₈; (C₂-C₁₀)alkynyl substituted with one or more substituents selected from the group consisting of —OH, —NO₂, cyano, —O—(C₁-C₁₀)alkyl, —C(O)OR₂₈, and —NR₂₈R′₂₈, provided that at least one of the substituents is —NR₂₈R′₂₈; and a known aromatic ring having 5 or 6 members, each one of the members being selected from the group consisting of —CRᵥ—, —N—, —O—, —NR′ᵥ, —S—;
Rₘ, R′ₘ, Rₜ and R′ₜ are the same or different and are selected from the group consisting of: H, (C₁-C₁₀)alkyl; (C₁-C₁₀)haloalkyl; (C₂-C₁₀)alkenyl; (C₂-C₁₀)alkynyl; (C₁-C₁₀)alkyl substituted with one or more substituents selected from the group consisting of —OH, —NO₂, cyano, —O—(C₁-C₁₀)alkyl, —C(O)OR₂₉, and —NR₃₀R′₃₀; and a known aromatic ring having 5 or 6 members, each one of the members being selected from the group consisting of —CR_{z}—, —N—, —NR′_{z}, —O—, and —S—;
at least one of Rᵥ, is —NR₃₁R′₃₁ and the other(s) Rᵥ are selected from the group consisting of H, —NR₃₁R′₃₁, (C₁-C₁₀)alkyl; (C₁-C₁₀)haloalkyl; (C₂-C₁₀)alkenyl; (C₂-C₁₀)alkynyl; and (C₁-C₁₀)alkyl substituted with one or more substituents selected from the group consisting of —OH, —NO₂, cyano, —O—(C₁-C₁₀)alkyl, —C(O)OR₃₂, and —NR₃₃R′₃₃;
R_{z}, and R′_{z} are independently selected from the group consisting of: H, (C₁-C₁₀)alkyl; (C₁-C₁₀)haloalkyl; (C₂-C₁₀)alkenyl; (C₂-C₁₀)alkynyl; (C₁-C₁₀)alkyl substituted with one or more substituents selected from the group consisting of —OH, —NO₂, cyano, —O—(C₁-C₁₀)alkyl, —C(O)OR₃₄, and —NR₃₅R′₃₅; and
R₂₈, R′₂₈, R₂₉, R₃₀, R′₃₀, R₃₁, R′₃₁, R₃₂, R₃₃, R′₃₃, R₃₄, R₃₅, and R′₃₅ are independently selected from the group consisting of: —H; (C₁-C₁₀)alkyl; (C₁-C₁₀)haloalkyl; (C₂-C₁₀)alkenyl; and (C₂-C₁₀)alkynyl;
to create a catecholamine membrane in the air/liquid interface in the absence of any support, the crosslinking reaction being performed in a liquid medium, wherein both the catechol and the amine are soluble at a pH comprised from 6.5 to 10, particularly from 6.5 to 8 and under agitation, particularly appropriate agitation; and
b) isolating the membrane resulting from step (a) from the air/liquid interface.

Worth to mention, through the process of the invention, the formation of the membranes takes place in the liquid-air interface, avoiding the use of substrates (e.g., glass, metals) or of the polymers which in situ provides the support (e.g., polyethyleneimine, PEI).

The agitation contributes to the continuous entrance of O₂ in the reaction medium, something that also determines the oxidation of the catechol derivative. And also allows for the diffusion of oxidized catechol moieties that crosslink with the amino-based ligands in the interface.

Regarding the aliphatic amine having a terminal amine at each end of the hydrocarbon chain of formula (II), it is shown below that when comparative tests were performed with an aliphatic monoamine having a terminal amine only at one end of the hydrocarbon chain (such as hexylamine), working under the particular reaction conditions of the invention provided a fragile membrane, which was not possible to isolate from the air/water interface.

Further advantages of the process of the invention are that it is cheap, eco-friendly, just involving two starting materials in aqueous medium, and avoids the use of harmful solvents.

Advantageously, the present inventors also found that aromatic amines of formula (IIbis) were also able to provide, when reacting with the catechol derivative, self-standing membranes.

The process of the invention allows for exquisite control of the resulting features. One of the characteristics that can be controlled is the final thickness of the membranes. This control can be easily achieved through the concentration and reaction time. The increase of the concentration of the starting reagents, together with higher reaction times, results in the formation of thicker membranes. With this control, it is possible to fabricate membranes with thickness ranging from 50 nm to 3 μm when measured by scanning electron microscopy (SEM) and atomic force microscopy (AFM), as provided below. Worth to mention that thicker membranes are easily handled compare with thinner membranes. Nevertheless, in all the cases was possible to isolate the floating membranes for their characterization and use (via functionalization).

The particular features of the process of the invention allow for the obtaining of a catecholamine-based membrane in the air/liquid interface. Therefore, the membrane obtainable by the process of the invention is a free-standing membrane (also so-called self-supported or self-standing membrane), which means that it does not require a support for the polymerization.

Not only that but also, the membrane resulting from the process of the invention shows some advantageous properties. As it is shown below, the membranes resulting from the process of the invention were robust, easy to handle and manipulate, highly flexible and adaptable to any kind of surface without breaking. In fact, they could be cut easily in different morphologies (e.g., squares, rectangles) using a sharp tool (e.g., scalpel or scissors) or produced in prefabricated molds with the desired morphology. Contrary to the membrane obtained from aliphatic monoamines having a terminal amine only at one end of the hydrocarbon chain which were so fragile in the absence of support that no formal membrane could be achieved, in spite of the efforts made by the inventors.

In addition, the free-standing membrane of the invention shows a Janus character. They exhibited not only a different roughness depending on the side but also asymmetric chemistry. Particularly, the present inventors found that the side in contact with liquid medium (in the examples, water) presented higher roughness because of the creation of a nanopatterning based on catecholamine nanoparticles (produced during the process of the invention), while the air-contact side was smoother. Surprisingly, the inventors found that once the membranes of the invention were formed and washed using ultrapure water, the nanopatterning of the liquid-side contact of the membranes remained invariable, i.e., the nanoparticles remained embedded in the surface. This unexpected nanopatterning in the liquid-contact side endows the membranes of the invention with a higher roughness surface. Worth to mention, this nanopatterning was not observed in the synthesized comparative membranes of PEI and with a substrate, where both sides had similar topography as observed by SEM. In these comparative examples, both sides presented similar surfaces without any different features between them.

Such morphological asymmetry property endows the membranes of the invention with added value as the liquid-contact (in the examples water-contact) side promotes a better cell adhesion compared with smoother surfaces observed in comparative synthesized membranes with PEI or using substrates, where the adhesion of cells was very low and its proliferation resulted in low density. In the case of the substrate, the membrane showed similar features on both sides with a roughness around 3.5 nm on both sides due to the substrate, which avoids the formation of nanoparticles that subsequently cannot be embedded on the surface, forming the nanopatterning.

Furthermore, self-supporting membranes of the prior art, obtained using PEI, cannot be used in contact with animals because they produce toxic by-products. Advantageously, the membrane of the invention is safe, being suitable to be used in contact with animal or human beings. As shown below, even degrading, the cells are able to adhere and grow on the membranes of the invention, contrary to the case wherein the membrane includes PEI (see examples below).

Altogether, the membrane of the invention shows improved features with respect to the catecholamine-based membranes reported in the prior art.

In view of the above, in a second aspect, the present invention provides a self-standing catecholamine-based membrane obtainable by the process of the first aspect of the invention.

As it is shown below, due to the particular properties of the membranes of the invention, they are particularly efficient in supporting the adhesion and differentiation of cells, which is indicative of its innate regenerative ability.

This is greatly due to the roughness and Janus character shown by the membranes of the invention, which, in addition, confers to the membrane of the invention the ability to be easily functionalized with a molecule of interest, either with a therapeutic profile or visualization means.

As it is shown below, the adhesion of cells is favoured in rough surfaces, thus enhancing their proliferation and growth. This property is of special interest for applying the membranes in tissue regeneration as they can be used as a platform for the growth of new tissue in damaged areas.

Therefore, in a third aspect, the present invention provides the catecholamine-based membrane as defined in the second aspect of the invention further comprising one or more therapeutic molecules for use in therapy, or alternatively, the catecholamine-based membrane as defined in the second aspect of the invention further comprising one or more detection labels for use in diagnosis.

A fourth aspect of the invention relates to the catecholamine-based membrane as defined in the second aspect of the invention, which further comprises one or more molecules of interest selected from the group consisting cells, growth factors and combinations thereof for use in regenerating tissues.

In a fifth aspect, the present invention provides the use of the catecholamine-based membrane as defined in the second aspect of the invention as an adhesive (for example in the form of a patch).

In a sixth aspect, the present invention provides the use of the catecholamine-based membrane as defined in the second aspect of the invention as a vehicle of a molecule of interest.

In a final aspect the present invention provides an article, such as a medical device and/or electronic parts (e.g., electrodes or sensors), which is partially or totally coated with the self-standing membrane of the second aspect of the invention.

### BRIEF DESCRIPTION OF DRAWINGS

FIG. 1 corresponds to SEM images of different membranes. In the images, it is possible to observe the different topography between the water-contact side (e and h) and the air-contact side (d and g). Besides, the flexibility of the membranes is corroborated by the lack of cracks or fissures. a) rolled membrane (1-7) showing the high flexibility, b) rolled membrane (3-7) with high flexibility, c) rolled membrane (2-7) showing the air-contact side; d) water-contact side of membrane (2-7), e) water-contact side of membrane (3-7), f) flexibility showed by the membrane (5-7) and with the air-contact side exposed, g) air-contact side of the membrane (1-11) and h) water-contact side of membrane (1-11).

### DETAILED DESCRIPTION OF THE INVENTION

All terms as used herein in this application, unless otherwise stated, shall be understood in their ordinary meaning as known in the art. Other more specific definitions for certain terms as used in the present application are as set forth below and are intended to apply uniformly throughout the specification and claims unless an otherwise expressly set out definition provides a broader definition.

For the purposes of the present invention, any ranges given include both the lower and the upper end-points of the range.

In a first aspect, the present invention provides a process for preparing a catecholamine-based membrane.

In the context of the present invention, the term “catecholamine-based membrane” and “catecholamine-based film” have the same meaning and can be interchangeably used and refers to a material made with a catechol derivative crosslinked with an amine compound. In the context of the invention, the catechol derivative is any compound including a moiety of formula (I), whereas the amine is an aliphatic amine of formula (II) or an aromatic amine of formula (IIbis). The crosslinking can be confirmed by any suitable routinary technique such as infrared (FT-IR) or UV-visible spectroscopy. As it is shown below, the inventors confirmed the successful cross-linking of the amine of formula (II) or (IIbis) with the catechol derivative by detecting covalent bonding formation between the catechol derivative and the amine-based ligand. This covalent interaction will depend on the functional groups of the catechol reacting with terminal amino groups. Usually, the polymerization of catechol-derivatives with amino-based molecules can be corroborated using FT-IR spectroscopy. For example, the decrease of intensity bands associated with hydroxyl groups, appearing in the range of 3600-3000 cm⁻¹, is a signal of copolymerization. Besides, the incorporation of amino-based ligands on the catechol rings forming the cross-linking can be confirmed through the appearance of new bands in the range of 1800-1650 cm⁻¹. Finally, the incorporation of aliphatic carbons present in amino-based ligands can be followed by detecting specific bands in the range of 3000-2500 cm⁻¹.

According to the present invention, the catechol derivative can be any including a moiety of formula (I). That is, the catechol derivative in the context of the invention is any compound comprising or consisting of a 6-membered aromatic ring, showing two vicinal hydroxyl groups (o-benzenediol). This catechol skeleton of formula (I) occurs in a variety of natural products such as drugs imitating them (such as MDMA), hormones/neurotransmitters, and catechin, which is found in tea. Many pyrocatechin derivatives have been suggested for therapeutic applications. Furthermore, there are many catechol derivatives commercially available from several companies (such as Sigma-Aldrich, Alfa Aesar and Fisher Scientific, among others). Illustrative non-limitative examples are pyrocatechol (CAS RN 120-80-9), pyrogallol (CAS RN 87-66-1), 4-methylcatechol (CAS RN 452-86-8), caffeic acid (CAS RN 331-39-5, 501-16-6, 71693-97-5), dopamine (CAS RN 51-61-6, 62-31-7), quercetin (CAS RN 117-39-5, 6151-25-3, 849061-97-8), hexahydroxy-triphenylene (CAS RN 4877-80-9), catechin (CAS RN 7295-85-4, 154-23-4, 18829-70-4, 225937-10-0), gallic acid (CAS RN 149-91-7, 5995-86-8), tannic acid (CAS RN 1401-55-4), and epigallocatechin gallate (CAS RN 989-51-5), among others.

In the context of the invention, the term “alkyl” (or “alkylene”, when referred to “B” biradical) refers to a saturated linear or branched hydrocarbon chain containing the number of carbon atoms indicated in the claims and in the description. Examples of alkyl groups include, but are not limited to: methyl, ethyl, propyl, iso-propyl, butyl, isobutyl, tert-butyl, pentyl, hexyl, heptyl, octyl, nonanyl, decanyl, and the like.

In the context of the invention, the term “alkenyl” (or “alkenylene”, when referred to “B” biradical) refers to a saturated linear or branched hydrocarbon chain containing the number of carbon atoms indicated in the claims and in the description and containing one or more double bond(s). Examples of alkenyl groups include, but are not limited to: ethenyl, propenyl, butenyl, 1-methyl-2-buten-1-yl, and the like.

In the context of the invention, the term “alkynyl” (or “alkynylene”, when referred to “B” biradical) refers to a saturated linear or branched hydrocarbon chain containing the number of carbon atoms indicated in the claims and in the description, and one or more triple bond(s). Examples of alkyl groups include, but are not limited to: ethynyl, 1-propynyl, 2-butynyl, 1,3-butadinyl, 4-pentynyl, and 1-hexynyl, and the like.

The terms “known heteroalkylene” and “known heteroalkenylene” refer to heteroalkylenes and heteroalkenylenes, which are known in the art and so intend to exclude those hetero systems that are not chemically possible.

In the context of the invention, the term “known 3 to 20-membered heteroalkylene” refers to any known saturated chain comprising from 3 to 20 atoms selected from carbons and heteroatoms (i.e., atoms other than carbon atoms, such as N, O or S), provided that the first and last atoms forming the unsaturated chain are carbon atoms (to which A and A′ are bound). For example, the member atoms are selected from the group consisting of —C(Rₓ)₂—, —CRₓ—, —N—, —NR′ₓ—, —S—, and —O—, provided that at least one of the members is —N—, —NRₓ—, —S—, or —O—. The heteroalkylene chain can be linear or branched. Rₓ is independently selected from the group consisting of —H; —OH; (C₁-C₁₀)alkyl; (C₂-C₁₀)alkenyl; (C₂-C₁₀)alkynyl; (C₁-C₁₀)haloalkyl; 0-(C₁-C₁₀)alkyl; —O—(C₂-C₁₀)alkenyl; —O—(C₂-C₁₀)alkynyl; nitro, —NRₓ₁Rₓ₂; (C₁-C₁₀)alkyl substituted with one or more substituents selected from the group consisting of —OH, —NO₂, cyano, —O—(C₁-C₁₀)alkyl, —C(O)OR₁₆, and —NR₁₇R′₁₇; and halogen. R_{X1}, Rₓ₂, R₁₆, R₁₇, R′₁₇, and R′ₓ are independently selected from the group consisting of —H, (C₁-C₁₀)alkyl, (C₂-C₁₀)alkenyl, (C₂-C₁₀)alkynyl, and (C₁-C₁₀)haloalkyl.

In the context of the invention, the term “known 3 to 20-membered “heteroalkenylene”, refers to any known unsaturated chain, including one or more double bonds, and made from 3 to 20 member atoms selected from carbons and heteroatoms (i.e., atoms other than carbon atoms, such as N, O or S), provided that at least the first and last atoms forming the chain are carbon atoms (to which A and A′ are bound). For example, the member atoms are selected from the group consisting of —C(Rₓ)₂—, —CRₓ—, —N—, —NR′ₓ—, —S—, and —O—, provided that at least one of the members is —N—, —NR′ₓ—, —S—, or —O—. The heteroalkylene chain can be linear or branched. Rₓ is independently selected from the group consisting of —H; —OH; (C₁-C₁₀)alkyl; (C₂-C₁₀)alkenyl; (C₂-C₁₀)alkynyl; (C₁-C₁₀)haloalkyl; O—(C₁-C₁₀)alkyl; —O—(C₂-C₁₀)alkenyl; —O—(C₂-C₁₀)alkynyl; nitro, —NRₓ₁Rₓ₂; (C₁-C₁₀)alkyl substituted with one or more substituents selected from the group consisting of —OH, —NO₂, cyano, —O—(C₁-C₁₀)alkyl, —C(O)OR₁₆, and —NR₁₇R′₁₇; and halogen. R_{X1}, Rₓ₂, R₁₆, R₁₇, R′₁₇, and R′ₓ are independently selected from the group consisting of —H, (C₁-C₁₀)alkyl, (C₂-C₁₀)alkenyl, (C₂-C₁₀)alkynyl, and (C₁-C₁₀)haloalkyl.

In the context of the invention, the term “haloalkyl” refers to a saturated linear or branched hydrocarbon chain containing the number of carbon atoms indicated in the claims and in the description, wherein at least one of the carbon atoms is substituted by at least one halogen.

The number of carbon atoms of an alkyl, alkenyl, alkynyl, akylene, alkenylene, and alkynylene, is represented by a “C” (symbol of the carbon atom) with a number in subindex format, which indicates the number of carbons. Thus, “C₁” means that the alkyl has a single carbon atom; and wherein reference is made to a range, in the form, for instance, of “(C₁-C₂₀)”, it means that the hydrocarbon has from 1 to 20 carbon atoms.

The term “known ring system” refers to a ring system, which is known in the art and so intends to exclude those ring systems that are not chemically possible.

According to the present invention a ring system formed by “isolated” rings means that the ring system is formed by two, three or four rings and said rings are bound via a bond from the atom of one ring to the atom of the other ring. The term “isolated” also embraces the embodiment in which the ring system has only one ring. Illustrative non-limitative examples of known ring systems consisting of one ring are those derived from: cyclopropyl, cyclobutyl, cyclopentyl, cyclohexyl, cycloheptyl, cyclopropenyl, cyclobutenyl, cyclopentenyl, phenyl, biphenylyl, and cycloheptenyl.

According to the present invention the expression “fused rings” encompasses rings totally fused, partially fused or spiro fused.

According to the present invention, when the ring system is “totally fused” it means that the ring system is formed by two, three or four rings in which two or more atoms are common to two adjoining rings. Illustrative non-limitative examples are 1,2,3,4-tetrahydronaphthyl, 1-naphthyl, 2-naphthyl, anthryl, or phenanthryl.

According to the present invention when the ring system is “partially fused”, it means that the ring system is formed by three or four rings, being at least two of said rings totally fused (i.e. two or more atoms being common to the two adjoining rings) and the remaining ring(s) being bound via a bond from the atom of one ring to the atom of one of the fused rings.

According to the present invention when the ring system is “spiro fused”, it means that the ring system comprises at least two rings sharing a common atom. The simplest spiro compounds are bicyclic (having just two rings), or have a bicyclic portion as part of the larger ring system, in either case with the two rings connected through the defining single common atom. Spiro compounds may be fully carbocyclic (all carbon) or heterocyclic (having one or more non-carbon atom forming part of the backbone of the rings).

In the context of the invention, the terms “halo” and “halogen” are used interchangeably and refer to a halogen group selected from the group consisting of chloro, fluoro, bromo and iodo.

In one embodiment, the catechol derivative is one of formula (III):

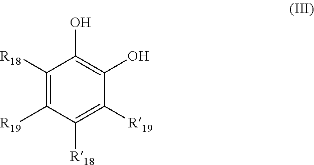

wherein
R₁₈, R₁₉, R′₁₈ and R′₁₉ are the same or different and are selected from the group consisting of:
   —H;
   —OH;
   —NR₂₀R′₂₀;
   halogen
   (C₁-C₁₀)alkyl;
   (C₂-C₁₀)alkenyl;
   (C₁-C₁₀)alkyl substituted with one or more substituents selected from the group consisting of: —OH, halogen, nitro, cyano, (C₁-C₁₀)alkyl, (C₁-C₁₀)haloalkyl, —NR₂₁R′₂₁, —C(O)OR₂₂, and —O—(C₁-C₁₀)alkyl;
   (C₂-C₁₀)alkenyl substituted with one or more substituents selected from the group consisting of: —OH, halogen, nitro, cyano, (C₁-C₁₀)alkyl, (C₁-C₁₀)haloalkyl, —NR₂₈R′₂₃, —C(O)OR₂₄, and —O—(C₁-C₁₀)alkyl;
   a known ring system consisting of one or two rings, each one of the rings: (a) consisting of 5 or 6 members selected from the group consisting of —C(R_{y})₂—, —CR_{y}—, —N—, —NR′_{y}, —S—, and —O—; (b) being saturated, partially unsaturated or aromatic, and (c) being isolated, partially isolated or fused;
   each one of the R_{y} is independently selected from the group consisting of —H, —OH, (C₁-C₁₀)alkyl, (C₂-C₁₀)alkenyl, (C₂-C₁₀)alkynyl, (C₁-C₁₀)haloalkyl, —O—(C₁-C₁₀)alkyl, nitro, —NR₂₅R₂₅, and halogen;
   R′_{y} is selected from the group consisting of —H, (C₁-C₁₀)alkyl, (C₂-C₁₀)alkenyl, (C₂-C₁₀)alkynyl, and (C₁-C₁₀)haloalkyl;
   R₂₀, R′₂₀, R₂₁, R′₂₁, R₂₃, R′₂₃, R₂₅, and R′₂₅ are the same or different and are selected from the group consisting of: —H; (C₁-C₁₀)alkyl; (C₁-C₁₀)haloalkyl; (C₂-C₁₀)alkenyl; (C₂-C₁₀)alkynyl; (C₁-C₁₀)alkyl substituted with one or more substituents selected from the group consisting of: —OH, halogen, nitro, cyano, (C₁-C₁₀)alkyl, (C₁-C₁₀)haloalkyl, —NR₂₆R′₂₆, —C(O)OR₂₇, and —O—(C₁-C₁₀)alkyl;
   R₂₂ and R₂₄ are independently selected from the group consisting of: H, (C₁-C₁₀)alkyl, (C₁-C₁₀)haloalkyl, (C₂-C₁₀)alkenyl; and (C₂-C₁₀)alkynyl; and
   R₂₆, R′₂₆ and R₂₇ are the same or different and are selected from the group consisting of: —H; (C₁-C₁₀)alkyl; (C₁-C₁₀)haloalkyl; (C₂-C₁₀)alkenyl; and (C₂-C₁₀)alkynyl.

In another embodiment, optionally in combination with any of the embodiments provided above or below, the catechol derivative of formula (III) is one wherein R₁₈ and R₁₉ are the same or different and are selected from the group consisting of: —H; —OH; (C₁-C₁₀)alkyl; (C₁-C₁₀)alkyl substituted as defined above; and (C₂-C₁₀)alkenyl substituted as defined above. In an alternative embodiment, optionally in combination with any of the embodiments provided above or below, the catechol derivative of formula (III) is one wherein one of R₁₈ and R₁₉ is a known ring system consisting of two rings, each one of the rings (a) consisting of 5-6 members selected from the group consisting of —C(R_{y})₂—, —CR_{y}—, —N—, —NR′_{y}—, —S—, and —O—, (b) being saturated, partially unsaturated or aromatic, and (c) being isolated, partially isolated or fused; wherein R_{y} and R′_{y} are as defined above. In an alternative embodiment, optionally in combination with any of the embodiments provided above or below, the catechol derivative of formula (III) is one wherein one of R₁₈ and R₁₉ is a known ring system consisting of two rings, each one of the rings (a) consisting of 6 members selected from the group consisting of —C(R_{y})₂—, —CR_{y}—, —N—, —NR′_{y}—, —S—, and —O—, (b) being saturated, partially unsaturated or aromatic, and (c) being fused; wherein R_{y} and R′_{y} are as defined above. In an alternative embodiment, optionally in combination with any of the embodiments provided above or below, the catechol derivative of formula (III) is one wherein one of R₁₈ and R₁₉ is a known ring system consisting of two rings, each one of the rings (a) consisting of 6 members selected from the group consisting of —C(R_{y})₂—, —CR_{y}—, —N—, —NR′_{y}—, —S—, and —O—, provided that at least one of the rings includes a heteroatom (—N—, —NR′_{y}, —S—, and —O—); (b) being saturated, partially unsaturated or aromatic, and (c) being fused; wherein R_{y} and R′_{y} are as defined above. In an alternative embodiment, optionally in combination with any of the embodiments provided above or below, the catechol derivative of formula (III) is one wherein one of R₁₈ and R₁₉ is a known ring system consisting of two rings, each one of the rings (a) consisting of 6 members selected from the group consisting of —C(R_{y})₂—, —CR_{y}—, —N—, —NR′_{y}—, —S—, and —O—, provided that at least one of the rings includes a —O— heteroatom; (b) being saturated, partially unsaturated or aromatic, and (c) being fused; wherein R_{y} and R′_{y} are as defined above. In an alternative embodiment, optionally in combination with any of the embodiments provided above or below, the catechol derivative of formula (III) is one wherein one of R₁₈ and R₁₉ is a known ring system consisting of two rings, each one of the rings (a) consisting of 6 members selected from the group consisting of —C(R_{y})₂—, —CR_{y}—, —O—, provided that at least one of the rings includes a —O-heteroatom; (b) being saturated, partially unsaturated or aromatic, and (c) being fused; wherein R_{y} and R′_{y} are as defined above. In an alternative embodiment, optionally in combination with any of the embodiments provided above or below, the catechol derivative of formula (III) is one wherein one of R₁₈ and R₁₉ is a known ring system consisting of two rings, each one of the rings (a) consisting of 6 members selected from the group consisting of —C(R_{y})₂—, —CR_{y}—, —O—, provided that only one of the rings includes a —O-heteroatom; (b) being saturated, partially unsaturated or aromatic, and (c) being fused; wherein R_{y} and R′_{y} are as defined above.

In one embodiment, optionally in combination with any of the embodiments provided above or below, the catechol of formula (III) is one wherein one of the R₁₈ or R₁₉ is —H and the other is as defined in any of the above embodiments.

In one embodiment, optionally in combination with any of the embodiments provided above or below, the catechol derivative of formula (III) is one wherein R′₁₈ and R′₁₉ are —H and R₁₈ and R₁₉ are as defined in any of the above embodiments.

In one embodiment, optionally in combination with any of the embodiments provided above or below, the catechol derivative is selected from the group consisting of pyrocatechol, dopamine, pyrogallol, caffeic acid, 4-methylcatechol, and catechin.

In another embodiment, optionally in combination with any of the embodiments provided above or below, the amine is a known aliphatic amine of formula (II).

In another embodiment, optionally in combination with any of the embodiments provided above or below, the amine is a known aliphatic amine of formula (II) wherein A and A′ are the same. In another embodiment, optionally in combination with any of the embodiments provided above or below, the amine is a known aliphatic amine of formula (II) wherein A and A′ are the same or different and represent NHR′₁. In another embodiment, optionally in combination with any of the embodiments provided above or below, the amine is a known aliphatic amine of formula (II) wherein A and A′ are the same and represent NHR′₁. In another embodiment, optionally in combination with any of the embodiments provided above or below, the amine is a known aliphatic amine of formula (II) wherein at least one of A and A′ represents —NH₂. In another embodiment, optionally in combination with any of the embodiments provided above or below, the amine is a known aliphatic amine of formula (II) wherein A and A′ are the same and represent —NH₂.

In another embodiment, optionally in combination with any of the embodiments provided above or below, the amine is a known aliphatic amine of formula (II) wherein B represents (C₁-C₂₀)alkylene; (C₁-C₂₀)alkylene substituted as defined above; or a known 3- to 20-membered heteroalkylene.

In another embodiment, optionally in combination with any of the embodiments provided above or below, the amine is a known aliphatic amine of formula (II) wherein B represents (C₁-C₂₀)alkylene; (C₁-C₂₀)alkylene substituted as defined above; or a known 3- to 20-membered heteroalkylene; and A and A′ are the same, such as —NH₂.

In another embodiment, optionally in combination with any of the embodiments provided above or below, the amine is a known aliphatic amine of formula (II) wherein B represents (C₁-C₁₅)alkylene; (C₁-C₁₅)alkylene substituted as defined above; or a known 3- to 15-membered heteroalkylene.

In another embodiment, optionally in combination with any of the embodiments provided above or below, the amine is a known aliphatic amine of formula (II) wherein B represents (C₁-C₁₅)alkylene; (C₁-C₁₅)alkylene substituted as defined above; or a known 3- to 15-membered heteroalkylene; and A and A′ are the same, such as —NH₂.

In one embodiment, optionally in combination with any of the embodiments provided above or below, the amine is a known aliphatic amine of formula (II) wherein B represents (C₁-C₁₅)alkylene, (C₂-C₁₅)alkylene, (C₄-C₁₅)alkylene or (C₅-C₁₅)alkylene.

In one embodiment, optionally in combination with any of the embodiments provided above or below, the amine is a known aliphatic amine of formula (II) wherein B represents (C₁-C₁₅)alkylene, (C₂-C₁₅)alkylene, (C₄-C₁₅)alkylene or (C₅-C₁₅)alkylene; and A and A′ are the same, such as —NH₂.

In another embodiment, optionally in combination with any of the embodiments provided above or below, the amine is a known aliphatic amine of formula (II) wherein B represents a known 3- to 15-membered heteroalkylene, particularly represents a known 3- to 10-membered heteroalkylene or a known 4- to 8-membered heteroalkylene. In another embodiment, optionally in combination with any of the embodiments provided above or below, the amine is a known aliphatic amine of formula (II) wherein B represents a known 3- to 15-membered heteroalkylene, particularly represents a 3- to 10-membered heteroalkylene; and A and A′ are the same, such as —NH₂. In another embodiment, optionally in combination with any of the embodiments provided above or below, the amine is a known aliphatic amine of formula (II) wherein B represents a known 3- to 15-membered heteroalkylene, particularly represents a 3- to 10-membered heteroalkylene, wherein the members are selected from carbon, S, and N atoms, as defined above. In another embodiment, optionally in combination with any of the embodiments provided above or below, the amine is a known aliphatic amine of formula (II) wherein B represents a known 3- to 15-membered heteroalkylene, particularly represents a 3- to 10-membered heteroalkylene, wherein each one of the members is selected from the group consisting of: C(Rₓ)₂, CRₓ, —NR′ₓ—, and —S—, wherein Rₓ and R′ₓ are as defined above.

In another embodiment, optionally in combination with any of the embodiments provided above or below, the amine is a known aliphatic amine of formula (II) wherein B represents a known 3- to 15-membered heteroalkylene, particularly represents a known 3- to 10-membered heteroalkylene, wherein the members are selected from carbon, S and N atoms as defined above; and A and A′ are the same, such as —NH₂.

In another embodiment, optionally in combination with any of the embodiments provided above or below, the amine is a known aliphatic amine of formula (II) wherein B represents a known 3- to 15-membered heteroalkylene, particularly represents a known 3- to 10-membered heteroalkylene, wherein two of the members are S atoms and the others are carbon atoms, as defined above. In another embodiment, optionally in combination with any of the embodiments provided above or below, the amine is a known aliphatic amine of formula (II) wherein B represents a known 3- to 15-membered heteroalkylene, particularly represents a known 3- to 10-membered heteroalkylene, wherein two of the members are S atoms and the others are carbon, as defined above; and A and A′ are the same, such as —NH₂. In another embodiment, optionally in combination with any of the embodiments provided above or below, the amine is a known aliphatic amine of formula (II) wherein B represents a known 4- to 15-membered heteroalkylene, particularly represents a known 4- to 10-membered heteroalkylene, wherein the members are carbon and S atoms, as defined above. In another embodiment, optionally in combination with any of the embodiments provided above or below, the amine is a known aliphatic amine of formula (II) wherein B represents a known 4- to 15-membered heteroalkylene, particularly represents a known 4- to 10-membered heteroalkylene, wherein the members are carbon and —NR′ₓ— atoms, as defined above. In another embodiment, optionally in combination with any of the embodiments provided above or below, the amine is a known aliphatic amine of formula (II) wherein B represents a known 4- to 15-membered heteroalkylene, particularly represents a known 4- to 10-membered heteroalkylene, wherein the members are carbon and S atoms, as defined above; and A and A′ are the same, such as —NH₂. In another embodiment, optionally in combination with any of the embodiments provided above or below, the amine is a known aliphatic amine of formula (II) wherein B represents a known 4- to 15-membered heteroalkylene, particularly represents a known 4- to 10-membered heteroalkylene, wherein the members are carbon and —NR′r atoms, as defined above; and A and A′ are the same, such as —NH₂. In another embodiment, optionally in combination with any of the embodiments provided above or below, the amine is a known aliphatic amine of formula (II) wherein B represents a known 4- to 15-membered heteroalkylene, particularly represents a known 4- to 10-membered heteroalkylene, wherein two of the members are S atoms and the remaining members are carbon atoms, as defined above. In another embodiment, optionally in combination with any of the embodiments provided above or below, the amine is a known aliphatic amine of formula (II) wherein B represents a known 4- to 15-membered heteroalkylene, particularly represents a known 4- to 10-membered heteroalkylene, wherein one of the members is —NR′ₓ— and the remaining members are carbon atoms, as defined above. In another embodiment, optionally in combination with any of the embodiments provided above or below, the amine is a known aliphatic amine of formula (II) wherein B represents a known 4- to 15-membered heteroalkylene, particularly represents a known 4- to 10-membered heteroalkylene, wherein two of the members are S atoms and the remaining members are carbon atoms, as defined above; and A and A′ are the same, such as —NH₂. In these embodiments wherein the heteroalkylene is one including two S atoms, the heteroalkylene can also be referred as “alkylene disulfide”. Illustrative non-limitative examples are cystamine, 4-aminophenyl disulfide and bis-amino polyethyleneglycol disulfide, among others.

In another embodiment, optionally in combination with any of the embodiments provided above or below, the amine is a known aliphatic amine of formula (II) wherein B represents a known 4- to 15-membered heteroalkylene, particularly represents a known 4- to 10-membered heteroalkylene, wherein one of the members is —NR′ₓ— and the remaining members are carbon atoms, as defined above; and A and A′ are the same, such as —NH₂.

In another embodiment, optionally in combination with any of the embodiments provided above or below, the amine is a known aliphatic amine of formula (II), wherein B represents a known 4- to 15-membered heteroalkylene, particularly represents a known 4- to 10-membered heteroalkylene, wherein each one of the members is selected from the group consisting of C(Rₓ)₂, CRₓ, —NR′ₓ—, and —S—, wherein Rₓ and R′ₓ are as defined above.

In another embodiment, optionally in combination with any of the embodiments provided above or below, the amine is a known aliphatic amine of formula (II) wherein B represents a known 3- to 15-membered heteroalkylene, particularly represents a known 3- to 10-membered heteroalkylene, wherein: (a) one or two of the members are —S— atoms and the others are carbon atoms selected from C(Rₓ)₂, and CRₓ; or, alternatively,
(b) one or two of the members are —NR′ₓ—, and the others are carbon atoms selected from the group consisting of C(Rₓ)₂, and CRₓ; wherein Rₓ is as defined above;
(c) one of the members is —S— atoms and the others are carbon atoms selected from the group consisting of C(Rₓ)₂, and CRₓ; or, alternatively,
(d) one of the members is —NR′ₓ—, and the others are carbon atoms selected from the group consisting of C(Rₓ)₂, and CRₓ;
(e) two of the members are —S— atoms and the others are carbon atoms selected from the group consisting of C(Rₓ)₂, and CRₓ; or, alternatively,
(f) two of the members are NR′ₓ—, and the others are carbon atoms selected from the group consisting of C(Rₓ)₂, and CRₓ;
wherein Rₓ and R′ₓ are as defined above.

In one embodiment, optionally in combination with any of the embodiments provided above or below, the known aliphatic amine of formula (II) is selected from the group consisting of an amine of formula (II1), an amino of formula (II2), and an amine of formula (II3):

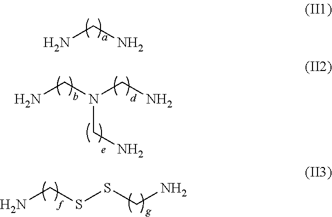

wherein a is an integer from 1 to 20; b, d, and e are independently an integer from 1 to 10, more particularly b, d, and e are the same; and f, and g are independently an integer from 1 to 10, more particularly f and g are the same.

In one embodiment, optionally in combination with any of the embodiments provided above or below, the known aliphatic amine of formula (II) is selected from the group consisting of hexamethylenediamine, octamethylenediamine, dodecamethylenediamine, cystamine, tris-(3-aminopropyl)amine, and tris-(2-aminopropyl)amine, more particularly selected from hexamethylenediamine, dodecamethylenediamine, octamethylenediamine and cystamine.

In another embodiment of the first aspect of the invention, optionally in combination with any of the embodiments provided above or below, the amine is an aromatic amine of formula (IIbis). In another embodiment of the first aspect of the invention, optionally in combination with any of the embodiments provided above or below, the amine is an aromatic amine of formula (IIbis1):

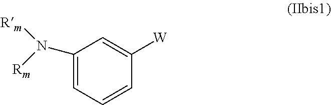

where Rₘ, R′ₘ and W are as defined above.

In another embodiment of the first aspect of the invention, optionally in combination with any of the embodiments provided above or below, the amine is an aromatic amine of formula (IIbis) or (IIbis1) which comprises at least two amino groups.

In another embodiment of the first aspect of the invention, optionally in combination with any of the embodiments provided above or below, the amine is an aromatic amine of formula (IIbis) or (IIbis1), and W represents —NRₜR′ₜ.

In another embodiment of the first aspect of the invention, optionally in combination with any of the embodiments provided above or below, the amine is an aromatic amine of formula (IIbis) or (IIbis1), W represents —NRₜR′ₜ, and Rₘ, R′ₘ, Rₜ and R′ₜ are the same. In another embodiment of the first aspect of the invention, optionally in combination with any of the embodiments provided above or below, the amine is an aromatic amine of formula (IIbis) or (IIbis1), W represents —NRₜR′ₜ, and Rₘ, R′ₘ, Rₜ and R′ₜ are the same and represent —H.

In another embodiment of the first aspect of the invention, optionally in combination with any of the embodiments provided above or below, the amine is an aromatic amine of formula (IIbis) or (IIbis1), W represents —NRₜR′ₜ, Rₘ and R′ₘ are the same and represent —H, and Rₜ and R′ₜ are the same and are as defined above.

In another embodiment of the first aspect of the invention, optionally in combination with any of the embodiments provided above or below, the amine is an aromatic amine of formula (IIbis) or (IIbis1), W represents —NRₜR′ₜ, and Rₜ and R′ₜ are the same and are other than hydrogen.

In another embodiment of the first aspect of the invention, optionally in combination with any of the embodiments provided above or below, the amine is an aromatic amine of formula (IIbis) or (IIbis1), W represents —NRₜR′ₜ, and Rₘ and R′ₘ are hydrogen; and Rₜ and R′ₜ are the same and are other than hydrogen.

In another embodiment of the first aspect of the invention, optionally in combination with any of the embodiments provided above or below, the amine is an aromatic amine of formula (IIbis) or (IIbis1), W represents —NRₜR′ₜ, Rₘ and R′ₘ are the same and represent —H, and Rₜ and R′ₜ represent an aromatic ring as defined above under the first aspect of the invention. In another embodiment of the first aspect of the invention, optionally in combination with any of the embodiments provided above or below, the amine is an aromatic amine of formula (IIbis) or (IIbis1), W represents —NRₜR′ₜ, Rₘ and R′ₘ are the same and represent —H, and each one of Rₜ and R′ₜ represent an aromatic ring having 6 members as defined above under the first aspect of the invention.

In another embodiment of the first aspect of the invention, optionally in combination with any of the embodiments provided above or below, the amine is an aromatic amine of formula (IIbis) or (IIbis1), W represents —NRₜR′ₜ, and each one of Rₜ and R′ₜ represent an aromatic ring as defined above.

In another embodiment of the first aspect of the invention, optionally in combination with any of the embodiments provided above or below, the amine is an aromatic amine of formula (IIbis) or (IIbis1), W represents —NRₜR′ₜ, and each one of Rₜ and R′ₜ represent an aromatic ring having 6 members as defined above.

In another embodiment of the first aspect of the invention, optionally in combination with any of the embodiments provided above or below, the amine is an aromatic amine of formula (IIbis) or (IIbis1), W represents —NRₜR′ₜ, and each one of Rₜ and R′ₜ represent an aromatic ring having 6 members, all the members being —CR_{z}—, wherein R_{z} is as defined above.

In another embodiment of the first aspect of the invention, optionally in combination with any of the embodiments provided above or below, the amine is an aromatic amine of formula (IIbis) or (IIbis1), W represents —NRₜR′ₜ, wherein Rₜ and R′ₜ are the same and represent an aromatic ring having 6 members, all the members, being —CR_{z}— members, wherein R_{z} is as defined above.

In another embodiment of the first aspect of the invention, optionally in combination with any of the embodiments provided above or below, the amine is an aromatic amine of formula (IIbis) or (IIbis1), W represents —NRₜR′ₜ, Rₘ and R′ₘ are the same and represent —H, and each one of Rₜ and R′ₜ represent an aromatic ring having 6 members, all the members being —CR_{z}—, wherein R_{z} is as defined above. In another embodiment of the first aspect of the invention, optionally in combination with any of the embodiments provided above or below, the amine is an aromatic amine of formula (IIbis) or (IIbis1), W represents —NRₜR′ₜ, Rₘ and R′ₘ are the same and represent —H, Rₜ and R′ₜ are the same and represent an aromatic ring having 6 members, all the members, being —CR_{z}—members, wherein R_{z} is as defined above.

In another embodiment of the first aspect of the invention, optionally in combination with any of the embodiments provided above or below, the amine is one of formula (IIbis) or (IIbis1), being W═NRₜR′ₜ, and one or more of Rₘ, R′ₘ, Rₜ and R′ₜ having the following formula (IV):

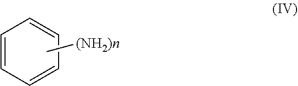

wherein n is 1 or 2, particularly 1, and the other(s) being —H.

Alternatively, in another embodiment of the first aspect of the invention, optionally in combination with any of the embodiments provided above or below, the amine is an aromatic amine of formula (IIbis) or (IIbis1), and W represents *S-S-L. In another embodiment of the first aspect of the invention, optionally in combination with any of the embodiments provided above or below, the amine is an aromatic amine of formula (IIbis) or (IIbis1), W represents *S-S-L, and Rₘ and R′ₘ are the same. In another embodiment of the first aspect of the invention, optionally in combination with any of the embodiments provided above or below, the amine is an aromatic amine of formula (IIbis) or (IIbis1), W represents *S-S-L, and Rₘ and R′ₘ are the same and represent —H. In another embodiment of the first aspect of the invention, optionally in combination with any of the embodiments provided above or below, the amine is an aromatic amine of formula (IIbis) or (IIbis1), W represents *S-S-L, Rₘ and R′ₘ are the same, and L represents an aromatic ring as defined above. In another embodiment of the first aspect of the invention, optionally in combination with any of the embodiments provided above or below, the amine is an aromatic amine of formula (IIbis) or (IIbis1), W represents *S-S-L, Rₘ and R′ₘ are the same and represent —H, and L represents an aromatic ring as defined above. In another embodiment of the first aspect of the invention, optionally in combination with any of the embodiments provided above or below, the amine is an aromatic amine of formula (IIbis) or (IIbis1), W represents *S-S-L, Rₘ and R′ₘ are the same, and L represents an aromatic ring having 6 members as defined above. In another embodiment of the first aspect of the invention, optionally in combination with any of the embodiments provided above or below, the amine is an aromatic amine of formula (IIbis) or (IIbis1), W represents *S-S-L, Rₘ and R′ₘ are the same and represent —H, and L represents an aromatic ring having 6 members as defined above. In another embodiment of the first aspect of the invention, optionally in combination with any of the embodiments provided above or below, the amine is an aromatic amine of formula (IIbis) or (IIbis1), W represents *S-S-L, Rₘ and R′ₘ are the same, and L represents an aromatic ring having 6 members, the same or different, represented by —CRᵥ—, as defined above. In another embodiment of the first aspect of the invention, optionally in combination with any of the embodiments provided above or below, the amine is an aromatic amine of formula (IIbis) or (IIbis1), W represents *S-S-L, Rₘ and R′ₘ are the same and represent —H, and L represents an aromatic ring having 6 members, the same or different, particularly the same, represented by —CRᵥ—, as defined above as defined above.

In another embodiment of the first aspect of the invention, optionally in combination with any of the embodiments provided above or below, the amine is an aromatic amine of formula (IIbis) or (IIbis1), and L represents an aromatic ring as defined above.

In another embodiment of the first aspect of the invention, optionally in combination with any of the embodiments provided above or below, the amine is an aromatic amine of formula (IIbis) or (IIbis1), and L represents an aromatic ring having 6 members as defined above.

In another embodiment of the first aspect of the invention, optionally in combination with any of the embodiments provided above or below, the amine is an aromatic amine of formula (IIbis) or (IIbis1), and L represents an aromatic ring having 6 members, the same or different, represented by —CRᵥ—, as defined above.

In another embodiment of the first aspect of the invention, optionally in combination with any of the embodiments provided above or below, the amine is an aromatic amine of formula (IIbis) or (IIbis1), and L has the following formula (IV):

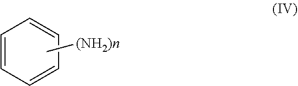

n 1 or 2, and one of the carbon atoms forming the aromatic ring being bound to the —S— atom. In another embodiment of the first aspect of the invention, optionally in combination with any of the embodiments provided above or below, the amine of formula (IIbis) is one of formula (V), (VI) or (VII):

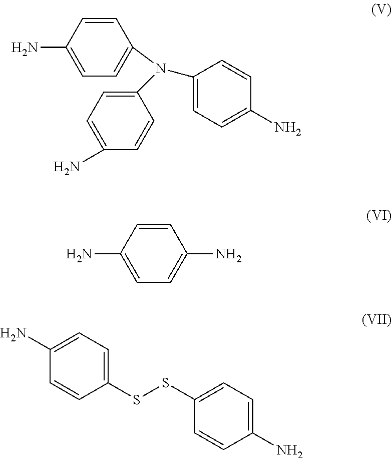 [SMILES: Nc1ccc(N(c2ccc(N)cc2)c2ccc(N)cc2)cc1 ; Nc1ccc(N)cc1 ; Nc1ccc(SSc2ccc(N)cc2)cc1]

In one embodiment, optionally in combination with any of the embodiments provided above or below, the amine is selected from the group consisting of: hexamethylenediamine, octamethylenediamine, dodecamethylenediamine, cystamine, tris-(3-aminopropyl)amine, tris-(2-aminopropyl)amine, and 4,4′,4″-triaminotriphenylamine.

The catechol derivative and the amine solutions can be separately prepared, and then being mixed together. Or, alternatively, one of the catechol derivative and the amine can be prepared in the form of a solution and the other one be added as directly obtained from the supplier.

The appropriate solvent system to prepare the solution(s) will depend on the polar nature of the catechol and amine, something which is part of the general knowledge of the skilled person in the art. Both, the catechol and the amine, have to dissolve in the same solvent system to carry out the crosslinking reaction. In one embodiment the catechol and amine are prepared in the form of an aqueous solution. The term “aqueous solution” embraces solutions consisting of only water but also combinations of water with other polar solvents such as water+alcohols or aqueous-based buffers. The aqueous solution is obtained by mere mixing of the compound with water. In any case, when the catechol and the amine are mixed, this step is performed under agitation (to appropriately promote the formation of the film in the air/liquid interface).

In one embodiment, optionally in combination with any of the embodiments provided above or below, the pH of the solution is comprised from 6.5 to 8 or from 7 to 7.5. In another embodiment, optionally in combination with any of the embodiments provided above or below, the pH is of 6.5, 6.6, 6.7, 6.8, 6.9, 7, 7.1, 7.2, 7.3, 7.4, 7.5, 7.6, 7.7, 7.8, 7.8, 7.9, 8, 8.1, 8.2, 8.3, 8.4, 8.5, 8.6, 8.7, 8.8, 8.9, 9, 9.1, 9.2, 9.3, 9.4, 9.5, 9.6, 9.7, 9.8, 9.9 or 10. There are well-known commercially available buffers that can be added the reaction medium to provide the intended pH value. Illustrative non-limitative examples are phosphate-buffered saline (PBS), carbonate-bicarbonate and citrate, among others. The buffer can be added in any order to the reaction medium with respect to the addition of catechol derivative and the amine.

In one embodiment, optionally in combination with any of the embodiments provided above or below, the liquid medium comprises an aqueous-based buffer, more particularly, an aqueous-based buffer in the absence of nitrogen atoms or amino groups, even more particularly, the aqueous-based buffer being selected from the group consisting of a phosphate buffer, a carbonate buffer and a citrate buffer.

In another embodiment, optionally in combination with any of the embodiments provided above or below, the liquid medium is water.

Once the pH was adjusted, the agitation is also adjusted. The agitation can be performed during the whole step of cross-linking. In this embodiment, as the skilled person would recognize, the speed of agitation may not provide turbulences as they would hindrance the appropriate formation of the membrane. Whether the speed is appropriate or not can be easily confirmed by the skilled person: if no turbulence is observed in the reaction medium, this will indicate that the speed is appropriate. If, when adjusted, the skilled person observed the onset of turbulences in the liquid medium, this will be indicative that the speed is not appropriate and that it should be reduced until said turbulences disappeared. Appropriate agitation means to be used during the whole cross-linking step are magnetic (or mechanical) means, using the lowest speed possible to avoid the turbulence in the air/liquid interface, being homogeneous during the whole procedure and avoiding erratic movement of the magnet or mechanical pieces involved in the agitation procedure. The skilled person can routinary determine the appropriate speed of agitation depending on the reactor's volume, liquid medium and means of agitation. For example, in case of the examples provided below, wherein the volumes were of about 20 mL, the inventors established as appropriate agitation one lower than 500 rpm using a magnetic stirrer. In one embodiment, optionally in combination with any of the embodiments provided above or below, the stirring speed is equal to or below than 450 rpm, equal to or below than 400 rpm, or equal to below than 350 rpm.

Alternatively, the agitation can be performed during part of the cross-linking reaction. In this embodiment the speed of agitation can be any: once the agitation is stopped, the reaction medium is left for a period of time such as the oxidized catechol moieties that crosslink with the amino-based ligands diffuse and arrange in the interface to form the membrane. In this alternative embodiment, the agitation can stop, for example, when a visual colour change is detected due to the oxidation of the catechol moieties. Then, it is left to complete the copolymerization reaction with the amino-based ligand, finishing with the formation of a floating membrane in the air/liquid interface.

In one embodiment, optionally in combination with any of the embodiments provided above or below, agitation is performed during the whole step of cross-linking or alternatively during part of the cross-linking reaction.

In one embodiment, optionally in combination with any of the embodiments provided above or below, agitation is performed during the whole step of cross-linking such that no turbulences are provided, more particularly at a stirring speed is equal to or below than 450 rpm, equal to or below than 400 rpm, or equal to below than 350 rpm.

In another embodiment, optionally in combination with any of the embodiments provided above or below, agitation is performed using the lowest speed possible to avoid the turbulence in the air/liquid interface, being homogeneous during the whole procedure and avoiding erratic movement of the magnet or mechanical pieces involved in the agitation procedure, more particularly at a stirring speed is equal to or below than 450 rpm, equal to or below than 400 rpm, or equal to below than 350 rpm.

In another embodiment, optionally in combination with any of the embodiments provided above or below, agitation is only performed during part of the cross-linking reaction, in particular agitation is performed such that no turbulences are provided until a change in coloration is observed and then it is stopped, even more particularly agitation is performed for a period of time from 10 min to 2 hours and then it is stopped. In another embodiment, optionally in combination with any of the embodiments provided above or below, the amine of formula (II) or (IIbis) is at a molar ratio excess with respect to the catechol derivative, particularly the molar ratio of the amine compound of formula (II) vs the catechol derivative is comprised from 1.1:1 to 3:1, particularly from 1.2:1 to 2:1.

In another embodiment, optionally in combination with any of the embodiments provided above or below, the crosslinking step is performed at a temperature comprised from 10 to 60° C. Particularly, at a temperature from 10 to 50° C., from 12 to 40° C. or from 15 to 38° C.

In another embodiment, optionally in combination with any of the embodiments provided above or below, the crosslinking step is performed for at least a period of 24 h. In one embodiment, optionally in combination with any of the embodiments provided above or below, step (a) is performed at a pH value from 7 to 7.5 and agitation equal to or below than 300 rpm. In another embodiment, optionally in combination with any of the embodiments provided above or below, step (a) is performed at a pH value from 7 to 7.5, agitation equal to or below than 300 rpm and the molar ratio of the amine compound of formula (II) vs the catechol derivative is comprised from 1.1:1 to 3:1, particularly from 1.2:1 to 2:1.

The skilled person is able to adjust routine parameters such as reaction time or concentration of reagents, which helps tuning the final thickness of the membrane.

Once the crosslinking step is finished and the catecholamine membrane has been created in the air/water or air/liquid interface in the absence of any support, it is isolated from the medium. This can be performed manually (at laboratory scale) using, for instance tweezers; or mechanically (at an industrial scale) using, for instance, ring-shaped tool.

In a second aspect the present invention provided a catecholamine-based membrane obtainable by the process of the invention defined above, either in the first aspect or any of the above particular embodiments.

Therefore, all the embodiments provided above, related to the process, are also embodiments of this second aspect of the invention.

The term “obtainable” and “obtained” have the same meaning and are used interchangeably. In any case, the expression “obtainable” encompasses the term “obtained”.

The chemical asymmetry shown by the membranes of the invention provides different functionalization options and a higher versatility as platforms as already explained above. Having different chemical groups on each side of the membrane allows for the anchoring of specific molecules depending on their activity. For example, the water-contact side, which is rougher, could be functionalized with growth factors, while the air-contact side (less rougher) could be functionalized with antibacterial moieties to avoid infections during the regeneration process. For this reason, as the exposed chemical groups in each side of the membrane are different, the versatility of these membranes allows for their functionalization with (bio)molecules of different chemical nature depending on the side.

Therefore, in one embodiment, the catecholamine-based film of the invention comprises one or more molecules of interest, such as a therapeutic molecule (e.g., peptides, proteins, or antibodies, such as an antibacterial moiety (e.g., cetrimide or silver nanoparticles) or a growth factor (e.g., FGF-2 or TGFβ 3), among others) or a detection label (e.g., calcein, nanoparticles, such as metal nanoparticles or antibodies), among others.

In one embodiment, optionally in combination with any of the embodiments provided above or below, the catecholamine-based membrane further comprises one or more molecules of interest selected from the group consisting of therapeutic molecules, cells, growth factors, detection labels (fluorescent active moieties, nanoparticles or antibodies), and combinations thereof.

The invention also provides a process for preparing the catecholamine-based membranes of the invention including one or more molecules of interest, the process comprising:
(i.1) performing step (a) of the process as defined in the first aspect of the invention or any of the embodiments of this aspect,
(i.2) adding to the reaction medium the molecule of interest; and
(i.3) performing step (b) of the process of the first aspect of the invention;
or, alternatively, the process comprising:
(ii.1) incubating the catecholamine-based membrane of the second aspect of the invention with the molecule of interest.
Step (i.2) is performed either directly adding to the reaction medium the molecule of interest as obtained from the supplier or in the form of an aqueous suspension (following manufacturer's instructions). The mild conditions used in the process of the invention advantageously allows the functionalization of the membrane in the own reaction medium wherein the membrane has been formed, which, advantageously, simplifies the process, reducing time and cost. As the membrane is formed on air/water or air/liquid interface, the adding step can be performed by pouring the solution in the reaction medium or, in case that the membrane is covering the whole surface of the reaction medium, by injecting the molecule's solution to the reaction medium through the membrane. The latter does not negatively affect to the integrity of the membrane, which is a further indicium of the robustness and improved mechanical properties of the membranes of the invention.

In the case of step (ii.1), the membrane has already been isolated from the interface. Therefore, the membrane may be incorporated to a medium comprising the solution with the molecule of interest (and the side in contact with the medium is functionalized) or, alternatively, the membrane is incorporated to a medium and a solution with the molecule is injected in the solution. The functionalization side can be selected previously, depositing the membrane with the desired side in contact with the liquid (aqueous) phase containing the molecule of interest. The membranes have functional groups exposed on the surface that can be used as anchoring points.

The functionalization of membrane's surface with the molecule of interest can occur by covalent bond, adsorption, or electrostatic interaction. In the case of a covalent bond, the functionalization can be performed through nucleophilic or electrophilic attack depending on the chemical nature of the molecule of interest. For example the presence of acyl chloride (e.g., stearoyl chloride) could act as electrophile in presence of amino-terminated groups. On the other hand, amino groups (e.g., hexadecamine) or thiol groups (e.g., 1,8-octanedithiol) could act as nucleophilic molecules being covalently bonded to the ring of catechol-derivatives. Besides, the presence of carboxylic acids in the backbone of the molecule of interest can be used for the formation of an amide bond (peptide-like bonding) with the terminal amino groups exposed on the surface of the membranes. Finally, the physical adsorption of molecules could be induced due to the entrapment of the molecule of interest in the backbone of the membranes. In one embodiment of the invention, the molecule of interest is covalently bound to the membrane surface of the second aspect of the invention.

In one embodiment, optionally in combination with any of the embodiments provided above or below, the catecholamine-based membrane of the invention further comprises one or more molecules of interest selected from the group consisting of therapeutic molecules, and detection labels (fluorescent active moieties, nanoparticles or antibodies). More particularly, the molecule of interest is covalently bound to the membrane, particularly by an amide bond.

The covalent bond can be achieved by any routine protocol, such as by generating an amide bound between the free amino groups of the membrane of the invention and the free carboxylic groups of the molecule of interest. The coupling reaction, in this case, can be performed using (1-ethyl-3-(3-dimethylamino) propyl carbodiimide, hydrochloride (EDC) and N-Hydroxysuccinimide (NHS). As a common procedure, the formed membranes can be introduced in Petri dishes of bigger diameter with a buffer within the range pointed out above. Then, the EDC is injected in the aqueous phase. After an appropriate period of time, such as 30 min, the active (bio)molecule (e.g., antibacterial moiety, florescent dye, growth factor, etc.) together with the NHS agent is injected in the aqueous phase. The reaction is stirred slowly, below 500 rpm, for a period of time enough to guarantee the covalent bond formation, such as 60 min. Then, the functionalized membranes can be extracted from the Petri dish and washed. The functionalization can be controlled and induced independently on both sides of the membranes, enhancing the chemical Janus behaviour of the membranes. Other catechol derivatives with different functional groups as terminal thiols, amines or acyl chloride can also be functionalized through the nucleophilic or electrophilic attack to the exposed functional groups on the membranes or the rings in the catechol-derivatives. The procedure would be similar as previously described. Some parameters such as the pH or the solvent can be routinely adjusted or selected for activating the reaction between the molecule of interest and the membrane.

As it has also been explained above and discussed in detail below, the particular Janus features of the membrane of the invention allows the efficient functionalization by both sides, which is a further distinctive trait with respect to the membranes already reported in the prior art.

The membrane of the invention as such shows improved cell adhesion when compared to the membranes of the prior art. This supports its usability as adhesive.

Not only that, but also, due to the particular Janus distribution, the membrane of the invention is also capable of being functionalized with different types of molecules, which supports its usability as a vehicle of a therapeutic or diagnostic molecule.

In addition to the above, and due to the particular properties of the material, the membrane of the invention can also be used as adhesive or to coat, due to such adhesive property, any article (such as a medical device, electronic support or sensor) requiring a catecholamine membrane. The membrane of the invention can be used as a part of other substrates or devices, endowing the final product with enhanced properties. For example, prosthesis could be totally o partially covered with the membranes for the enhancement of biocompatibility and reducing side effects or organism rejection. Additionally, sensors or electronical supports could be adhered to biological tissues or devices through the use of the membranes of the invention. The sensors or electronic supports could be as a part of the membranes in a single platform or forming a hybrid composite, where the sensor or electronical support is adhered on the desired area with the help of the membranes applied as adhesive patches.

Throughout the description and claims the word “comprise” and variations of the word, are not intended to exclude other technical features, additives, components, or steps. Furthermore, the word “comprise” encompasses the case of “consisting of”. Additional objects, advantages and features of the invention will become apparent to those skilled in the art upon examination of the description or may be learned by practice of the invention. The following examples are provided by way of illustration, and they are not intended to be limiting of the present invention. Furthermore, the present invention covers all possible combinations of particular and preferred embodiments described herein.

### EXAMPLES

### 1. Materials

The catechol-based compounds (pyrocatechol(1), caffeic acid(2), dopamine(3), 4-methylcatechol(4), pyrogallol(5), and catechin(6)) as well as the amine compounds of formula (II) (hexamethylenediamine(7), octamethylenediamine(8), dodecamethylenediamine(9), cystamine(10), tris-(3-aminopropyl)amine(11), tris-(2-aminopropyl)amine(12) and 4,4′,4″-Triaminotriphenylamine(13)), and the comparative hexylamine and polyethylenimine (PEI) were purchased from Sigma-Aldrich (Merck, Madrid, Spain) and used without further purification. Type 1 ultrapure water from a Milli-Q filtration system (Millipore, Burlington, MA, USA) was used in all experiments unless otherwise specified. In some experiments, different water-based buffers were used: phosphate-buffered saline (PBS, pH 7.4, Sigma-Aldrich) and aqueous carbonate buffer (pH 9.1) were prepared, dissolving 1.89 g of NaHCO₃ and 265 mg of Na₂CO₃ in 250 mL of Milli-Q water.

### 2. Methods

### 2.1 Physicochemical Characterization

Fourier Transform Infrared (FT-IR) spectroscopy. The FT-IR spectra were recorded by using a Tensor 27 FTIR spectrometer (Bruker Optik, GmbH, Berlin, Germany), equipped with a room temperature detector and a mid-IR source (4000 to 400 cm⁻¹).
A) Characterization of the reagents by depositing the solid sample on a window reflection of diamond Attenuated Total Reflectance (ATR, model MKII Golden Gate, Specac). This method was used to characterize the reagents' IR.
B) Characterization of the membranes. Hyperion 2000 FTIR microscope (Bruker Optik, GmbH, Ettlingen, Germany) was used in reflection mode, which is equipped with a nitrogen-cooled mercury-cadmium-telluride (MCT) detector (InfraRed Associates, Inc., Stuart, FL, USA) using a 15x reflection objective, a gold mirror as reference and scanning for 1 min with a resolution of 4 cm⁻¹. For the analysis, the sample was deposited on a gold surface.

All the data obtained were processed using the Opus version 7.2.139.1294 (Bruker) software. Gold-coated glass substrates for FT-IR analysis were provided.

X-Ray Photoelectron Spectroscopy (XPS). Measurements were performed with a Phoibos 150 analyzer (SPECS EAS10P GmbH, Berlin, Germany) in ultra-high vacuum conditions (based pressure 10⁻¹⁰ mbar, residual pressure around 10⁻⁷ mbar). Monochromatic Al Kα line was used as X-ray source (1486.6 eV and 300 W). The electron energy analyzer was operated with pass energy of 50 eV. The hemispherical analyzer was located perpendicular to the sample surface. The data was collected every eV with a dwell time of 0.5 s. A flood gun of electrons, with energy lower than 20 eV, was used to compensate for the charge. The samples were deposited on silicon substrates. All the data was treated with CasaXPS version 2.3.17PR1.1 (Casa Software LTD, Teignmouth, UK) and OriginPro version 8.0988 (OriginLab Corporation, Northampton, MA, USA) software.

Scanning Electron Microscopy (SEM). The images were acquired by using scanning electron microscopy (SEM) (FEI Quanta 650 FEG, Thermo Fisher Scientific, Eindhoven, The Netherlands) in secondary electron mode with a beam voltage of 20 kV and a chamber pressure of 10⁻⁵ Pa. The working distance was set at 10 mm and different magnifications were tested for final images. The samples were prepared by deposition of the free-standing membranes on aluminium stubs. Before performing the analysis, the samples were metalized by depositing on the surface a thin platinum coating (5 nm) using a sputter coater (Leica EM ACE600). Aluminium-tapped supports (pin stubs) were provided.

Atomic Force Microscopy (AFM). Surface topography imaging of the different samples was carried out in ambient air in tapping mode using beam shaped silicon cantilevers (Nanosensors, nominal force constant: 5 N·m⁻¹, tip radius: −7 nm) on an Agilent 5500 AFM/SPM microscope (Keysight Technologies, Santa Clara, CA, USA) combined with PicoScan5 version 1.20 (Keysight Technologies) software. An external X-Y positioning system (closed-loop, 12 NPXY100E from nPoint, USA) was used. Image processing was done using open source software: WSxM version 3.1 (Nanotec Electronica, Madrid, Spain) and Gwyddion version 2.46 (CMI, Brno, Czech Republic).

Contact Angle (CA). Static contact angle measurements were performed with an EasyDrop contact angle meter (KRÜSS GmbH, Hamburg, Germany) using 15 μL water droplets. Each membrane was measured at three different points for obtaining an average of the whole surface. The measurements were performed approximately one minute after the droplet deposition.

Optical and Fluorescence Microscopy (OM). Images were obtained in transmittance mode with Zeiss Axio Observer Z1m (Carl Zeiss AG, Jena, Germany). The membranes were attached on glass substrates. The optical images were taken in bright field and the fluorescent images were acquired by changing between the different fluorescent probes DAPI and Alexa 488.

UV-vis spectroscopy. Ultraviolet-visible spectroscopy (UV-vis) was performed using a Cary 4000 UV-vis spectrometer (Agilent Technologies, Santa Clara, CA, USA) within range wavelengths from 200 to 800 nm and a 1 cm path length quartz cuvette (QS 10 mm). The baseline was corrected using a blank sample of pure solvent. All the measurements were taken under atmospheric conditions.

2.2 Biological characterization

a) Cell culture. Different cell lines were used for the testing of the membranes: human cervical carcinoma cells HeLa (ATCC® CCL-2), fibroblast cell line NIH/3T3 (ATCC® CRL-1658), adipose-derived mesenchymal ASC cells (ATCC® PCS-500-011) were purchased from LGC Standards S.L.U. (Barcelona, Spain). The base media (DMEM, Glutamax and glucose) were all supplemented with 10% fetal bovine serum, 100 μL/mL penicillin and 100 pg/mL streptomycin (Life Technologies, Thermo Fisher Scientific, Waltham, MA, USA). All cell cultures were maintained in a humidified incubator at 37° C. under an atmosphere of 5-10% CO₂.

b) Degradation of the membranes. The stability of the membranes in mouse plasma was determined at different time intervals over 3 months. For this purpose, the membranes were incubated with mouse plasma at pH 7.4 (BiolVT, Hicksville, NY, USA) at 37° C. The samples were then placed in Sectra/Por® dialysis tubing with a molecular weight cut-off (MWCO) of 1-3 kDa (Spectrum, New Brunswick, NJ, USA) and dialyzed against 20 mL of 1×PBS at a pH 7.4. Three aliquots were taken at predefined time points (5 min, 10 min, every remaining 10 min up to 1 h, every 30 min up to 10 h, 12 h, 16 h, 20 h, 1 day, 2 days, 3 days, 5 days, 7 days and every week after completing 3 months) and measured by UV-vis spectroscopy. The measurements were focused on the detection of a band in the range of 250-450 nm, corresponding to the signal associated with catechol moieties. The appearance of bands in the aforementioned region as well as their growth could be associated with the progressive degradation of the membranes. The liquid extractions obtained from the outside of the dialysis bag were tested in vitro using different cell lines (HeLa and NIH/3T3) to determine the cytotoxicity of potential side products, coming from the degradation of the membranes.

c) In vitro cell viability studies. Cells were added to a 48-well plate at a density of 15000 cells per well and allowed to adhere for 24 h. The different types of membranes cut in discs of 0.8 mm were then added to the wells in quadruplicate and the plate was returned to the incubator. MTT and PrestoBlue™ assays for cell respiration were carried after 24 h and 72 h of incubation.
(i) For the MTT assay, 20 μL of 5 mg/mL thiazoyl blue tetrazolium bromide (MTT, Sigma-Aldrich) solution was added to each well and incubated for 2 h. The supernatant was aspirated without disturbing the cells, and 150 μL of dimethyl sulfoxide (DMSO) was added to each well to dissolve the formazan salts from the metabolism of MTT in metabolically active cells. After complete dissolution, the absorbance was read at 540 nm using a BioTek MX plate reader (BioTek Instruments Inc., Winooski, VT, USA). The percentage of cell viability was calculated by dividing the absorbance of each well by the corresponding value of the control wells only treated with vehicle (1×PBS). A minimum of four independent experiments were performed on different days.
(ii) For the PrestoBlue™ assay, 10 μL of resazurin was added to the media to a final concentration of 15 μM, and the fluorescence was read using a Victor3 plate reader (PerkinElmer, Waltham, MA, USA) with an excitation wavelength of 531 nm and emission of 572 nm. As with the MTT assay, a minimum of four independent experiments were performed and cell viability was calculated against vehicle-treated cells.

d) Adhesion, proliferation and cell differentiation. The adhesion of the cells on the surface membranes was tested for all the aforementioned cell lines. Membranes were cut in discs of 0.8 mm using a sterile biopsy punch and placed in a 48-well plate with PBS. Then, the PBS was removed, allowing for the membrane to cover the bottom of the well. The membranes were seeded with the cells (25000 cells/membrane) added in 50 μl of the medium. Subsequently, the membranes were incubated for 30 min and after this time, the wells were filled with 350 μl of medium. The membranes with the cells were incubated for different times: 1 day, 3 days, 7 days, 14 days and 30 days.

After each time point, the membranes were washed for at least five times with PBS to eliminate the non-adhered cells. Different criteria were stablished in order to evaluate the adhesion, proliferation and cell differentiation for each cell type and membranes. For the adhesion, the membranes were washed in PBS for at least five times in order to remove unattached cells. Besides, the cells on the surface were counted using an image analysis software (ImageJ), counting the cells attached in the two different sides and at different time points, determining the density of cells. The proliferation was evaluated by analysing with optical and/or electronic microscopy the formation of cell networks and their preferential growth in specific directions as occurred in common in vitro cell culture. Finally, the cell differentiation was evaluated for the ASC cells, identifying the formation of different cell types through the observation of different morphology and cell structure using optical and/or electronic microscopy. For the experiments of cell differentiation with ASC cell line, the medium was enriched with growth factors (TGFβ 3 and BMP 6). For the observation by fluorescent and electronic microscopy, the cells were treated with formaldehyde for 1 h and subsequently washed three times with PBS. The membranes with cells were stored in the fridge with PBS. In the case of fluorescent imaging, the cells were previously stained with a Live/Dead kit test (Thermo Fisher Scientific) following the specifications indicated by the manufacturer. Specifically, for SEM analysis, the PBS was removed and then washed with increasing EtOH solutions (30%, 50%, 70%, 80%, 96% and 100%). Subsequently, the cells were washed three times with hexamethyldisilazane and left to dry at room temperature overnight. Finally, the membranes with the cells were mounted in SEM stubs for their image acquisition.

e) In vivo assays

Animal studies were approved by the local ethics committee, according to the regional and state legislation. For the assays, Sprague Dawley rats (12 male and 12 female) were purchased (Envigo, Indianapolis, IN, USA). First of all, the in vivo adhesion of the membranes was tested. To do this, the membranes were placed in the knee joint, drying excess blood and other fluids with gauze. The membranes of the invention remained adhered. Subsequently, the wound was sutured and the implanted membranes were checked at 3 and 7 days after the rats were euthanized. In another experiment, a joint injury was induced in rats to remove cartilage and adhere the membranes directly to bone, checking their adherence in vivo. These experiments were performed (n=5) with functionalized and non-functionalized membranes and with and without cells (ASC cell line) on the membranes. On the other hand, a tolerability test was carried out by implementing the membranes subcutaneously and in muscle to observe possible inflammation, hematoma or erythrema. These tests were performed for 4 and 8 days, checking the aspect of the skin visually (observation of irritation or redness) and once euthanized by observing some damaged effect in the surrounding tissues. All the tests were carried out for different formulations of the membranes of the invention and that PEI-based for comparative purposes.

### 3. Synthesis of the Membranes of the Invention

The corresponding catechol- and amine-derivative were placed in solid in the reaction vessel, the latter being in a molar ratio excess of the amine (1:1.5). Then, the addition of PBS was performed and the pH of the solution was adjusted to 7.4. The reaction vessel was covered with Parafilm® with a hole of 2 mm in diameter, allowing for the entrance of oxygen. The formation of the free-standing floating membranes was performed under magnetic stirring at 300 rpm, and at room temperature. The polymerization reaction took place in the liquid-air interphase through the oxidation of the catechol-derivatives and its reaction with the amine-based ligands, without the need of physical substrate or the formation of in situ substrates with other secondary molecules. After 24 h, the free-standing floating membranes were ready to be isolated. The membranes used in the in vitro and in vivo below tests were synthesized in a biological safety cab (Biosafety Class II Cabinet Telstar BioVanguard) to guarantee sterile conditions (avoiding air contaminants).

Once the membranes were synthesized they were treated with absolute ethanol overnight.

Table 1 summarizes the reagents and IR characterization.

TABLE 1

| | Reagents | IR characterization |
|---|---|---|
| (1-7) | Pyrocatechol | 3250, 2850, 1700, 1500, 1457 |
| | Hexamethylenediamine | 1260, 987 |
| (1-8) | Pyrocatechol | 3275, 2810, 1723, 1499, 1268, 856 |
| | Octamethylenediamine | |
| (1-9) | Pyrocatechol | 3249, 2842, 1715, 1623, 1519, |
| | Dodecamethylenediamine | 1261, 1110, 951 |
| (1-10) | Pyrocatechol | 3232, 1872, 1755, 1508, 1327, |
| | Cystamine | 1260, 741 |
| (1-11) | Pyrocatechol | 3349, 2632, 1791, 1611, 1547, |
| | Tris(3-aminopropyl)amine | 1281, 1027, 821 |
| (1-12) | Pyrocatechol | 3250, 2853, 1705, 1511, 1263, |
| | Tris(2-aminopropyl)amine | 1190, 808 |
| (2-7) | Caffeic acid | 3345, 2761, 1701, 1546, 1270, 981 |
| | Hexamethylenediamine | |
| (2-8) | Caffeic acid | 3221, 2747, 1703, 1628, 1515, |
| | Octamethylenediamine | 1260, 1134, 911 |
| (2-9) | Caffeic acid | 3271, 2623, 1731, 1718, 1635, |
| | Dodecamethylenediamine | 1334, 1210, 801 |
| (2-10) | Caffeic acid | 3251, 1711, 1595, 1409, 1261, 849 |
| | Cystamine | |
| (2-11) | Caffeic acid | 3331, 2617, 1793, 1529, 1405, |
| | Tris(3-aminopropyl)amine | 1315, 1039, 767 |
| (2-12) | Caffeic acid | 3253, 2821, 1712, 1538, 1211, |
| | Tris(2-aminopropyl)amine | 1204, 817 |
| (3-7) | Dopamine | 3261, 2777, 1678, 1511, 1231, |
| | Hexamethylenediamine | 1000, 897 |
| (3-8) | Dopamine | 3255, 2781, 1712, 1321, 1231, |
| | Octamethylenediamine | 1001, 877 |
| (3-9) | Dopamine | 3249, 2671, 1678, 1309, 1252, |
| | Dodecamethylenediamine | 1091, 810 |
| (3-10) | Dopamine | 3222, 1892, 1702, 1564, 1251, |
| | Cystamine | 1099, 897 |
| (3-11) | Dopamine | 3252, 2861, 1789, 1661, 1452, |
| | Tris(3-aminopropyl)amine | 1209, 818 |
| (3-12) | Dopamine | 3341, 2817, 1702, 1611, 1509, |
| | Tris(2-aminopropyl)amine | 1236, 981 |
| (4-7) | 4-methylcatechol | 3245, 2891, 1763, 1652, 1567, |
| | Hexamethylenediamine | 1251, 1171, 826 |
| (4-8) | 4-methylcatechol | 3240, 2761, 1890, 1624, 1351, |
| | Octamethylenediamine | 1246, 1171, 845 |
| (4-9) | 4-methylcatechol | 3265, 2871, 1873, 1763, 1597, |
| | Dodecamethylenediamine | 1250, 912 |
| (4-10) | 4-methylcatechol | 3252, 1802, 1720, 1583, 1247, 835 |
| | Cystamine | |
| (4-11) | 4-methylcatechol | 3241, 2732, 1709, 1432, 1267, |
| | Tris(3-aminopropyl)amine | 1161, 764 |
| (4-12) | 4-methylcatechol | 3249, 2801, 1527, 1341, 1223, |
| | Tris(2-aminopropyl)amine | 1127, 982 |
| (5-7) | Pyrogallol | 3367, 2791, 1654, 1328, 1219, |
| | Hexamethylenediamine | 1100, 879 |
| (5-8) | Pyrogallol | 3259, 2745, 1762, 1530, 1276, 811 |
| | Octamethylenediamine | |
| (5-9) | Pyrogallol | 3247, 2891, 1703, 1672, 1451, |
| | Dodecamethylenediamine | 1230, 932 |
| (5-10) | Pyrogallol | 3327, 1893, 1706, 1583, 1201, |
| | Cystamine | 1009, 873 |
| (5-11) | Pyrogallol | 3250, 2893, 1763, 1456, 1261, 801 |
| | Tris(3-aminopropyl)amine | |
| (5-12) | Pyrogallol | 3328, 2781, 1781, 1509, 1233, 834 |
| | Tris(2-aminopropyl)amine | |
| (6-7) | Catechin | 3246, 2893, 1703, 1576, 1240, |
| | Hexamethylenediamine | 1094, 932 |
| (6-8) | Catechin | 3267, 2871, 1705, 1521, 1277, |
| | Octamethylenediamine | 1127, 872 |
| (6-9) | Catechin | 3231, 2872, 1653, 1430, 1284, |
| | Dodecamethylenediamine | 1191, 899 |
| (6-10) | Catechin | 3243, 1654, 1398, 1255, 1132, 873 |
| | Cystamine | |
| (6-11) | Catechin | 3276, 2878, 1769, 1432, 1209, |
| | Tris(3-aminopropyl)amine | 1098, 918 |
| (6-12) | Catechin | 3255, 2873, 1872, 1643, 1587, |
| | Tris(2-aminopropyl)amine | 1432, 1210, 1154, 874 |
| (1-13) | Pyrocatechol | 3329, 1690, 1500, 1327, 1257, |
| | 4,4′,4″- | 1175, 1120, 912, 827 569, 506 |
| | Triaminotriphenylamine | |

### 4. Synthesis of Membranes for Comparative Purposes

Membranes for comparative purposes were also synthesized following the protocol provided above but modifying one of the reagents and/or reaction conditions to those pointed out in Table 2 below:

TABLE 2

| | Comparative membranes | | |
|---|---|---|---|
| Membrane | Reagents | Parameters | FT-IR bands |
| PEI-3 | Polyethyleneimine (PEI) | pH 11, no stirring, | 3447, 2982, 1640, 1193, 712 |
| | Dopamine | water | |
| Substrate | Pyrocatechol | SiO₂ substrate, pH 7.4, | 3250, 2850, 1700, 1500, 1457 |
| | Hexamethylenediamine | no stirring | 1260, 981 |
| Aliphatic | All catechol derivatives | pH 7.4, 300 rpm, water | 3265, 2763, 1675, 1110, 912 |
| Monoamine | Hexylamine | | |

### 5. Functionalization of the Membranes

The (bio)molecules were anchored through a coupling reaction using (1-ethyl-3-(3-dimethylamino) propyl carbodiimide, hydrochloride (EDC) and N-Hydroxysuccinimide (NHS). This coupling reaction allows for the formation of amide bonds between the amino groups exposed in the membranes and the carboxylic groups of the (bio)molecules functionalized. As a common procedure, the formed membranes were introduced in Petri dishes of bigger diameter compared with the membranes and containing PBS and under magnetic stirring (500 rpm). Then, an aqueous solution of EDC (5 mM) was injected in the aqueous phase. After 30 min, the active (bio)molecule together with the NHS agent were injected in the aqueous phase. The antibacterial moiety (vanillic acid) or the fluorescent dye (calcein) were dissolved in water (10 mM) and subsequently mixed with NHS (10 mM).) The reaction was stirred slowly for 60 min. Then, the functionalized membranes were isolated from the Petri dish, washed for at least 5 times with a flux of distillated water and dried under vacuum. This approach allowed for different functionalization with high yield (approx. 55-60%, depending on the functionalized molecule), thanks to the membranes composition, which are rich in amino groups exposed on the surface. The functionalization can be controlled and induced independently on both sides of the membranes, enhancing the chemical Janus behaviour of the membranes. The membranes were paced first

Different functionalizations with this approach were performed, as a representative example, two specific cases are presented below.

### 6. Results and Discussion

### 6.1. Mechanical Properties of the Membranes

The membranes of the invention were easily isolated by using common tweezers and properly handled and translated to other containers for their storage. The resulting membranes presented high flexibility without breaking when one end of the membranes was bent 1800 putting into contact with the other end of the membrane. This was performed in both humid and dried membranes without observation of any crack or fissure by SEM. This bending movement was performed manually for at least 50 cycles corroborating the flexibility of the membranes. Additionally, torsion movements were performed to check the flexibility limits of the membrane, showing excellent robustness.

Firstly, it was confirmed that the membrane was a catecholamine-based membrane through several measures using FT-IR and XPS. Depending on the chemical nature of the catechol derivative and the amine-based ligand, different frequency ranges of FT-IR spectra were evaluated. Firstly, the presence of catechol (3500-3000 cm⁻¹) and amine (1700-1500 cm⁻¹) groups was the first indication of the formation of the crosslinking polymer. Subsequently, depending on the type of catechol or amine-based molecules, specific bands were identified within certain ranges. For example, bands corresponding to an amide bond appeared in the range of 1650-1500 cm⁻¹ and between 1290-1200 cm⁻¹ specifically in 1-7 (1500 and 1260 cm⁻¹), 1-8 (1499 and 1268 cm⁻¹), 1-9 (1519 and 1261 cm⁻¹), 1-10 (1508 and 1260 cm⁻¹), 1-11 (1547 and 1281 cm⁻¹), 1-12 (1511 and 1263 cm⁻¹), 2-7 (1546 and 1270 cm⁻¹), 2-8 (1515 and 1260 cm⁻¹), 2-9 (1635 and 1210 cm⁻¹), 2-10 (1595 and 1261 cm⁻¹), 2-11 (1529 cm⁻¹), 2-12 (1538 and 1204 cm⁻¹), 3-7 (1511 and 1231 cm⁻¹), 3-8 (1231 cm⁻¹), 3-9 (1252 cm⁻¹), 3-10 (1564 and 1251 cm⁻¹), 3-11 (1209 cm⁻¹), 3-12 (1509 and 1236 cm⁻¹), 4-7 (1567 and 1251 cm⁻¹), 4-8 (1246 cm⁻¹), 4-9 (1597 and 1250 cm⁻¹), 4-10 (1583 and 1247 cm⁻¹), 4-11 (1267 cm⁻¹), 4-12 (1527 and 1223 cm⁻¹), 5-7 (1219 cm⁻¹), 5-8 (1530 and 1276 cm⁻¹), 5-9 (1230 cm⁻¹), 5-10 (1583 and 1201 cm⁻¹), 5-11 (1261 cm⁻¹), 5-12 (1509 and 1233 cm⁻¹), 6-7 (1576 and 1240 cm⁻¹), 6-8 (1521 and 1277 cm⁻¹), 6-9 (1284 cm⁻¹), 6-10 (1654 and 1255 cm⁻¹), 6-11 (1209 cm⁻¹) and 6-12 (1587 and 1210 cm⁻¹), the imine in the range of 1700-1600 cm⁻¹, specifically in 1-7 (1700 cm⁻¹), 1-9 (1623 cm⁻¹), 1-11 (1611 cm⁻¹), 2-7 (1701 cm⁻¹), 2-8 (1628 cm⁻¹), 2-9 (1635 cm⁻¹), 3-7 (1638 cm⁻¹), 3-9 (1678 cm⁻¹), 3-11 (1661 cm⁻¹), 3-12 (1611 cm⁻¹), 4-7 (1652 cm⁻¹), 4-8 (1624 cm⁻¹), 4-12 (1627 cm⁻¹), 5-7 (1654 cm⁻¹), 5-9 (1672 cm⁻¹), 6-7 (1703 cm⁻¹), 6-8 (1705 cm⁻¹), 6-9 (1653 cm⁻¹) and 6-12 (1643 cm⁻¹), and the incorporation of aliphatic carbons in the range of 3000-2500 cm⁻¹, specifically in 1-7 (2850 cm⁻¹), 1-8 (2810 cm⁻¹), 1-9 (2842 cm⁻¹), 1-11 (2632 cm⁻¹), 1-12 (2853 cm⁻¹), 2-7 (2761 cm⁻¹), 2-8 (2747 cm⁻¹), 2-9 (2623 cm⁻¹), 2-11 (2617 cm⁻¹), 2-12 (2821 cm⁻¹), 3-7 (2777 cm⁻¹), 3-8 (2781 cm⁻¹), 3-9 (2671 cm⁻¹), 3-11 (2861 cm⁻¹), 3-12 (2817 cm⁻¹), 4-7 (2891 cm⁻¹), 4-8 (2761 cm⁻¹), 4-9 (2817 cm⁻¹), 4-11 (2732 cm⁻¹), 4-12 (2801 cm⁻¹), 5-7 (2791 cm⁻¹), 5-8 (2745 cm⁻¹), 5-9 (2891 cm⁻¹), 5-11 (2893 cm⁻¹), 5-12 (2781 cm⁻¹), 6-7 (2893 cm⁻¹), 6-8 (2871 cm⁻¹), 6-9 (2872 cm⁻¹), 6-11 (2878 cm⁻¹) and 6-12 (2873 cm⁻¹), among others. In the case of membranes synthesized using amino-based ligands with aromatic moieties (e.g., 1-13), the incorporation of rings to the copolymer structure was detected through the appearance of specific band at 2765 cm⁻¹. Besides, the bonding between catechol-ligand and the exposed amino groups from the aromatic-based ligand was confirmed though the appearance of two new bands at 1500 and 1327 cm⁻¹. Finally, the shift of the bands at 1175 and 1120 cm⁻¹ was attributed to the new bonding formation with aromatic terminal-amino groups. Worth to mention, the incorporation of catechol and amino moieties to the resulting crosslinking structure was confirmed by the bands in the range of 3500-3000 cm⁻¹ and 1700-1500 cm⁻¹, respectively.

Later with XPS, it is confirmed that the chemical environments of the molecules have been modified, thus confirming the formation of specific bonds due to the copolymerization between catechol- and amino-derivative molecules. For this reason, high-resolution measurements were performed in C, N and 0 signals and the percentage of CNOH determined, thus indicating the final composition of the membranes. Worth to mention, in all the membranes of the invention, the amount of coexisting quinone/hydroxyl groups was higher in the water-contact side, compared with amino-terminal groups, which were predominant in the air-contact side. This differentiated functional groups exposition depending on the contact side could be of interest at the time of functionalization, which each side could be selected for the anchoring of specific molecules.

Contrary to the membranes of the invention:
PEI-based membrane required high pH (ca. 11); showed very low flexibility due to the formation of breaks when manipulated and bended and a plastic-like appearance due to the smooth and bright appearance of the resulting membrane. Besides, topographic AFM measurements highlighted the low roughness in both sides. Showing a low adaptability of the membrane to the surface;
membrane coating substrate: although the synthesis on the substrate was successful, the detachment from the substrate turned to be very difficult due to the high adhesion to the substrate and the brittle character of the formed membrane. Only small fragments were possible to take out from the surface, thus hampering their further bioapplication. For this reason, de manipulation and handle of the membranes performed on the substrate were more difficult and less efficient compared with the membranes of the invention; and
Using an aliphatic monoamine having a terminal amine only at one end of the hydrocarbon chain, no membrane was obtained in spite of the attempts performed.

### 6.2. Morphological Characterization (Optical Microscopy, SEM, AFM)

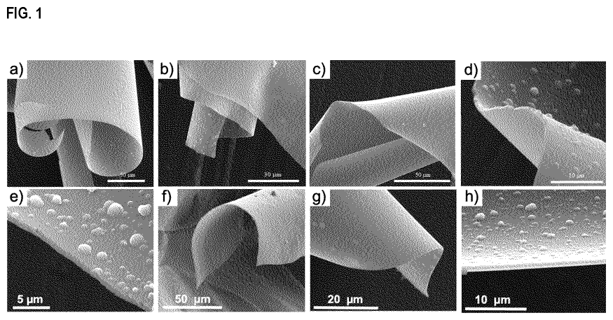

The free-standing membranes of the invention exhibited Janus character in terms of morphology. When studying their topography by SEM, both sides of the films were found to be different. The surface in contact with the water had embedded nanoparticles of the same cathecol-amine material. These nanoparticles were formed as a side product and were found as precipitate and embedded in the water-contact side. The precipitated NPs were centrifuged and isolated for their characterization by FT-IR, corroborating its catechol-amine composition. Besides, the formation of the membranes was followed through the time, taking aliquots form the interface at different time points and observed by SEM. The SEM images showed the process through the NPs were embedded. On the contrary the side exposed to air did not showed the embedded NPs, it was smooth and clean. The analysis of the images obtained with SEM (FIG. 1) highlighted the formation of membranes with different surface patterning. As explained in the following sections, this patterning influences in the final adhesion of the membranes to biological tissues but also to the cells.

It is also remarkable that once the membranes of the invention were formed, and washed using ultrapure water, the nanopatterning of the water-side contact of the membranes remained invariable, i.e., the nanoparticles remained embedded in the surface. This unexpected nanopatterning in the water-contact side endow the membranes with a higher roughness surface, which favor the cell adhesion (as further shown below).

Worth to mention, this nanopatterning was not observed in the synthesized comparative membranes of PEI and with substrate, where both sides had similar topography as observed by SEM. In these comparative examples, both sides presented similar surface without any different feature between them.

In particular, PEI-based membrane showed a chemical Janus character as PEI was perfectly separated within the formed structure forming two different domains and different porosity depending on the side. However, no nanopatterning was detected. Due to the plastic-like morphology (rigidity and smooth sides) exhibited by PEI membranes the roughness of the PEI-based membrane was very low in both sides when analyzed SEM images, presenting similar characteristics. This low roughness and lack of nanopatterning difficult the adhesion of cells on the membrane, as discussed in the following sections.

Regarding the membrane obtained from a substrate, this comparative membrane did not show any Janus character as both sides of the membrane were very similar, as the use of a substrate avoid the formation of a floating membrane in the interphase (membranes of the invention) and the ordering of the molecules due to their polarity on the water and air phase cannot take place.

In order to study in detail the surface of the free-standing membranes, further studies with SEM and AFM were carried out. Firstly, SEM was used for the analysis of the thickness of the membranes of the invention (Table 3). Worth to mention, the increase of the reaction time resulted in an increase of the thickness of the membranes. One explanation of this effect is due to the increase of amount of polymerized material through the time. Secondly, AFM was performed in order to determine the roughness of some of the membranes (Table 4) by measuring the topography properties in both air- and water-contact sides.

Additionally, AFM topographic profiles were evaluated to verify the thickness measurements obtained by SEM.

As a qualitative observation, SEM images showed different roughness depending on the contact side of the membranes. Specifically, water-contact side presented an unexpected higher roughness. For this reason, AFM was performed on the water-contact side. The membranes of the invention exhibited nanoparticles embedded, offering the highest roughness to the surface. Several areas were explored with regular profiles from different zones with a height of around 275 nm. Regarding the morphology, the nanoparticles embedded in the membrane correlated with SEM observations. In the case of the air-contact side, a smoother surface was found, with roughness less than 10 nm. Finally, the profile height match within the interval of thickness estimated by SEM, being in the range of 200-600 nm, depending on the membrane.

Finally, the roughness of both sides for the different membranes was measured as shown in Table 4 below.

TABLE 3
Thickness (nm)

| Membrane | 10 mM 24 h | 10 mM 48 h |
|---|---|---|
| (1-7) | 145 | 1065 |
| (1-8) | 223 | 2432 |
| (1-9) | 156 | 2456 |
| (1-10) | 324 | 3234 |
| (1-11) | 235 | 4378 |
| (1-12) | 276 | 3012 |
| (2-7) | 145 | 3340 |
| (2-8) | 254 | 2489 |
| (2-9) | 276 | 2082 |
| (2-10) | 109 | 3001 |
| (2-11) | 265 | 4567 |
| (2-12) | 243 | 3789 |
| (3-7) | 145 | 2987 |
| (3-8) | 143 | 2913 |
| (3-9) | 109 | 2375 |
| (3-10) | 187 | 3468 |
| (3-11) | 199 | 4728 |
| (3-12) | 201 | 5652 |
| (4-7) | 143 | 4765 |
| (4-8) | 121 | 4978 |
| (4-9) | 108 | 5091 |
| (4-10) | 155 | 4234 |
| (4-11) | 132 | 5675 |
| (4-12) | 156 | 5238 |
| (5-7) | 175 | 4456 |
| (5-8) | 139 | 2283 |
| (5-9) | 109 | 2487 |
| (5-10) | 111 | 2109 |
| (5-11) | 155 | 3108 |
| (5-12) | 143 | 3287 |
| (6-7) | 154 | 2278 |
| (6-8) | 127 | 3783 |
| (6-9) | 122 | 4873 |
| (6-10) | 101 | 3321 |
| (6-11) | 231 | 5523 |
| (6-12) | 221 | 3675 |
| (6-13) | 352 | 4983 |

Thickness values for the membranes of the invention at different reaction times at 10 mM of amine-based ligand concentration.

TABLE 4

Roughness (nm)

| Membrane | Water side | Air side |
|---|---|---|
| (1-7) | 33 | 6 |
| (1-8) | 36 | 4 |
| (1-9) | 45 | 3 |
| (1-10) | 34 | 6 |
| (1-11) | 65 | 7 |
| (1-12) | 34 | 3 |
| (3-7) | 25 | 5 |
| (3-8) | 33 | 4 |
| (3-9) | 41 | 2 |
| (3-10) | 23 | 3 |
| (3-11) | 32 | 6 |
| (3-12) | 45 | 4 |
| (4-7) | 35 | 5 |

Interestingly, the side in contact with water presented higher roughness because of the nanoparticles embedded, while the air-contact side is smoother. This property endows the membranes with added value as the water-contact side will promote a better cell adhesion compare with smoother surfaces, as observed in comparative synthesized membranes with PEI, where the adhesion of cells was very low and its proliferation resulted in low density (see below). Besides, the membrane synthesized using a substrate showed similar features on both sides with a roughness around 3.5 nm in both sides. This could be due to the fact that the membrane was synthesized in contact with a substrate that avoids the formation of nanoparticles that subsequently cannot be embedded on the surface forming the nanopatterning.

To sum up, morphological results proved that the membranes of the invention have Janus character from a morphological point of view. By these superficial techniques, SEM and AFM, not only different topographies, depending on the side, were shown, but also roughness was evaluated. Moreover, the thickness was measured by SEM and AFM for each membrane type. In all the cases, similar results were obtained, which demonstrate the reproducibility and universality of our approach for the formation of catecholamine-based membranes.

Finally, the membranes were characterized using optical microscopy. This was performed due to the observed interaction of light with the surface of the membranes, which was expressed in the reflection of different colors depending on the thickness of the surface. This phenomenon confirmed both the different thicknesses of the membranes and the nanopatterning on the surfaces.

The above was confirmed by determining the FT-IR, XPS and contact angle for each case. This characterization was performed for all the membranes of the invention and the comparatives. In all the cases similar results were obtained.

The results of FT-IR spectroscopy gave information of species present in the membranes, a read of the whole material in order to confirm its chemical composition.

The spectra were analyzed and shared the following characteristics. The broad band in the range of 3500-3000 cm⁻¹, corresponding to the stretching vibrations of hydroxyl (—OH) and in the range of 1700-1500 cm⁻¹ for amine (—NH₂) groups, whose peaks could be appreciated in the catechol derivatives and amine-based compound spectra, respectively. The peaks in the range of 3000-2500 cm⁻¹ are associated with asymmetric and symmetric stretching vibrations of aliphatic carbons (C—H). The peak at 1700 cm⁻¹ corresponded to the presence of quinones (C═O). The signal in the range of 1500-1400 cm⁻¹ can be assigned to C═C—H and C═C vibrations from the catecholic/quinonic rings of the catechol derivatives. The peaks in the range of 1200-1450 cm⁻¹ belongs to a secondary amine binding an alkyl or an aromatic ring.

Therefore, the chemical composition of the membranes matches the functional groups of the reagents. In all the combinations done, the FT-IR spectra showed similar results with the presence of specific bands for the amino and catechol groups. Additionally, for each specific catechol-derivative, other representative signals depending on the functional groups of the molecules can be assigned. These results also confirmed the successful copolymerization between the catechol- and amino-based molecules following the established methodology.

Finally, the wettability of the membranes was tested using contact angle equipment. Previous to performing the measurements, the membranes were placed in an oven at 170° C. for 2 h to completely dry them and stored under vacuum to remove all the water molecules. This drying process was followed by weighting the membranes until the lowest weight was reached, thus indicating the loss of all the water. With these measurements, the hydrophobic/hydrophilic character of the membranes was determined. As observed in other characterization techniques, the wettability was different depending on the side where it was measured. The water-contact side has a more hydrophilic behaviour with contact angles around 250, while the air-contact side has higher angles around 40°, denoting higher hydrophobicity. Nevertheless, overall the membranes show an intermediated hydrophilic/hydrophobic behaviour. This surface property could be changed by anchoring hydrophobic or hydrophilic moieties by using the exposed functional groups present on the membranes. Playing with the wettability, the adhesion of the membranes can be adjusted depending on the final tissue to be applied. For example, having more hydrophilic character of the membranes in the water-contact side (were more cells can growth), favour the adhesion of the membrane directly to the damaged tissue, while having a more hydrophobic character on the air-contact side, will avoid undesired adhesion with other tissues in de surrounding area.

### 6.3. Functionalization

### 6.3.1. With Antibacterial Molecules

One of the main problems, when a biomaterial has to be applied in medicine, is the risk of infection, this becoming especially relevant in the regenerative processes. For this reason, incorporating antibacterial moieties into the membranes can enhance the regeneration process of the damaged tissue without side-effects.

Thus, the inventors functionalized the membranes with vanillic acid, which has a well-known antibacterial activity, through the EDC/NHS coupling reaction, following the procedure described above. The tests were performed as a proof-of-concept in six different membranes (1-7, 1-8, 2-7, 2-8, 3-9, 3-10) In order to confirm the covalent attachment, the membranes were characterized by different means. FT-IR spectra confirmed the appearance of a band at 1665 cm⁻¹ assigned to the amide bond formation through the free amino groups and the carboxylic acid of the vanillic acid. Furthermore, specific peaks from the vanillic acid were incorporated in the spectra corresponding to the functionalized membranes.

Additional XPS measurements showed significant changes in the spectra corresponding to C1s and O1s, when comparing non- and functionalized membranes. These results corroborated the incorporation of the antibacterial moiety to the membranes. Worth to mention, the functionalized membranes were strongly washed in order to remove any unreacted material.

Finally, contact angle measurements were performed on the surface of non- and functionalized membranes (Table 5). The results highlighted an increase in the contact angle values once the membranes were functionalized. These measurements indirectly indicate that the surface membrane has been modified.

TABLE 5

Contact angle (°)

| Membrane | Non-functionalized | Functionalized |
|---|---|---|
| (1-7) | 25.6 | 38.3 |
| (1-8) | 27.8 | 40.1 |
| (1-9) | 22.6 | 39.1 |
| (1-10) | 25.8 | 38.5 |
| (1-11) | 29.5 | 41.3 |
| (1-12) | 23.5 | 37.4 |
| (3-7) | 28.1 | 39.5 |
| (3-8) | 30.2 | 42.4 |
| (3-9) | 21.2 | 38.3 |
| (3-10) | 28.4 | 41.3 |
| (3-11) | 23.3 | 37.9 |
| (3-12) | 20.5 | 35.8 |

### 6.3.2. With Fluorescent Moieties

Another interesting functionality for endow added value to the membranes is the functionalization with fluorescent molecules that will help to its monitoring and visualization in with used bioimaging techniques.

Thus, the membranes (1-7, 1-8, 2-7, 2-8, 3-9, 3-10) were functionalized with a fluorescent dye (calcein). The anchoring of the dye to the membrane was achieved via EDC/NHS coupling reaction, following the procedure described above. After the functionalization synthesis, the membranes were characterized using a fluorescent microscope.

It was found that the membranes were successfully functionalized with the fluorescent dye. The anchoring through the coupling reaction was stable and remained invariable even for several weeks in both dried and submerged membranes, as no loss of fluorescent signal was observed by fluorescent microscopy. These tests were performed by maintaining the functionalized membranes under water and measuring the fluorescence at different time points during at least 4 weeks. Additionally, the fluorescence of completely dried membranes and stored under vacuum, was also checked within the same period. The robust functionalization and the retention of the fluorescent activity was corroborated by measuring fluorescence with a) fluorescent microscopy and b) following the delivery of the dye by a dialysis experiment. For this last, degradation tests were performed placing the membranes in dialysis tubing bag (Spectrum® with pore size of 4-6 kDa) under magnetic stirring (500 rpm) and at room temperature. Different aliquots at different time points were taken from the outside of the dialysis bag and measuring them in a fluorometer. The fluorescent spectra showed that the functionalized membranes were stable in water with no significant dye released after 3 months due to the lack of the emission band associated to the dye (calcein, λₑₘᵢₛₛᵢₒₙ=521 nm)

Worth to mention, the as-functionalized membranes of the invention presented very high fluorescence with a time exposition of around 450 ms, which was enough for the acquisition of the images without saturation of the signal. Additionally, as in the case of antibacterial moieties, FT-IR spectra measurements corroborated the functionalization of the membrane with the fluorescent dye, appearing the typical bands corresponding to the calcein reagent (1750, 1630, 1445 and 1280 cm⁻¹). Besides, contact angle measurements showed an increase of water contact angle of around 300 after functionalization of the membranes, thus indicating a successful modification of the surface.

### 6.4. Biological Characterization

### 6.4.1. Degradation

The degradability of all the membranes of the invention was tested in physiological media (PBS, pH 7.4 and at 37° C.) using dialysis experiments. At different time points, aliquots from the outside of the dialysis bag were taken and measured in UV-vis to detect if the band corresponding to the catechol groups appeared (in the range between 250-450 nm depending on the catechol-derivative).

This monitoring was performed for 4 months, and, remarkably, no signal from catechol was measured. This means that the membranes of the invention are robust and do not degrade significantly. This stability is essential for their application in tissue regeneration in order to have the membrane for all the regenerative processes. The membranes retain its structure enough time for promoting the regeneration of the tissue.

Worth to mention, the extractions (aliquots) taken from the dialysis experiments were also tested in vitro in different cells lines. The results demonstrated that no cytotoxic subproducts were released from the membranes during their degradation process.

Contrary to the membranes of the invention, the PEI-based membrane of the prior art degraded remarkably faster when compared with the membranes of the invention, detecting traces of PEI after 2 weeks in a dialysis experiment. This delivery of the PEI affected the robustness of the membrane. Not only this, but also PEI reported toxicity in biological environments, being highly cytotoxic in different cell lines. This toxicity hampered the application of PEI-based membranes for tissue regeneration and other bioapplications.

### 6.4.2. Cell Viability

All the membranes of the invention, both functionalized and non-functionalized, have been tested in vitro in all the cell lines mentioned in the above Experimental Section. The results showed excellent biocompatibility with no toxicity even for the longer times of more than 14 days. The cells were able to grow, proliferate and differentiate (the latter using the case of ASC cells). As mentioned before, the extractions taken from the degradability assays during 4 months, were also tested in the different cell lines. The tests were performed for 72 h, showing that no toxic products were delivered.

Worth to mention, the cells were growing and proliferating until full covering of the membranes without any signs of membrane-induced cell death.

Additionally, the PEI-based membrane was tested. The results showed higher cytotoxicity compared with the membranes of the invention. After 24 h, all the membranes of the invention showed cell viability higher than 90% in all the cell lines tested. However, PEI-based membranes showed an increased toxicity with cell viability around 65%. This higher toxicity has been associated with the fast degradation of PEI-based membrane, inducing the release of PEI which has a well-known cytotoxic effect. This is one of the main reasons why the PEI-based membranes cannot be used in biological applications.

### 6.4.3. Cell Adhesion and Proliferation

The different cell lines were seeded on the membranes placed on the bottom of the cell culture plates and incubated with cell culture medium at different time points until 21 days. At the different time points, the membranes were extracted and washed for their characterization.

It was found that, in the case of the membranes of the invention, the cells were perfectly adhered to the membrane surface (both sides) with a preferential growth in the side in contact with water which was the one with the nanopatterning. This preferential growth was observed with a higher cell density on the water-contact side compared with the air-contact side (an increase of the 45%). Different images were taken with optical and electronic microscopy. The cells adhered on the membranes through the same mechanisms observed in common cell culture plate, showing their natural cell morphology and proliferation in preferred orientations.

The cells were marked with fluorescent moieties to observe them using fluorescent microscopy and also cells were fixed for their observation in SEM. The membranes were washed to remove non-attached cells.

Finally, PEI-based membranes showed very poor cell adhesion with very low density of cells. When compared with the membranes of the invention, the cell density on PEI-based membranes decreased around 70%. This could be associated, together with the toxic nature of PEI, to the surface characteristics of the membranes made with this polyimine, that hamper the interactions between the material and the cells.

### 6.4.4. Ex Vivo Adhesion

The adhesion of the membranes was also tested in ex vivo tissues (cartilage and skin from pig). The tissues were obtained directly from euthanized animals for other purposes used by the Veterinary Faculty of the Autonomous University of Barcelona. The objective was to corroborate the adhesion of the membranes in different types of biological tissues and in humid conditions. For this, different membranes of the invention (1-7, 1-8, 1-9, 1-10, 1-11, 1-12, 2-7, 2-8, 2-9, 2-10, 2-7, 2-8, 2-9, 2-10, 2-11, 2-12, 3-7, 3-8, 3-9, 3-10, 4-7, 4-8), were cut to different sizes and attached to the tissues. The tests were performed with non- and functionalized membranes and membranes with and without cells. Using gauze, the area could be cleaned without affecting the adhered membrane, which remained in the area without moving. In order to test the adhesion in extreme conditions, the biological tissues with the adhered membranes were immersed in water for months, observing that the membrane remained adhered to the tissue without detaching form the tissue.

### 6.4.5. In Vivo

The in vivo assays were performed with Dawley Sprague rats of female and male sex. Seven membranes of different composition were tested (1-7, 1-12, 2-7, 3-7, 3-11, 5-1 and 5-11) with and without ASC cells (see above).

The membranes were placed in cartilage, bone and muscle, showing excellent adhesion to the tissue with no signal of rejection. Besides, no inflammation, erythema and edema were observed. These preliminary results demonstrated the excellent in vivo tolerability and biocompatibility.

For comparative proposes, the membrane based on PEI was tested, showing very low adhesion to biological tissues due to its very low roughness and lack of interactions between the membrane and the tissue. Besides, its lack of flexibility made it difficult to apply it correctly, resulting in the membrane not being able to cover the biological tissue adequately. Due to its low adhesion to the tissue and rigidity, it was impossible to continue with the test and it had to be withdrawn.

### CITATION LIST

### Non Patent Literature

lacomino M. et al., “Multifunctional Thin Films and Coatings from Caffeic Acid and a Cross-Linking Diamine”, 2017, Langmuir, 33, 9, 2096-2102 (doi: 10.1021/acs.langmuir.6b04079);
Ponzio F. et al., “Polydopamine deposition at fluid interfaces”, 2016, Society of Chemical Industry, pages 1-7 (doi 10.1002/pi.5124); and
Suarez-Garcia S. et al., “Copolymerization of a Catechol and a Diamine as a Versatile Polydopamine-Like Platform for Surface Functionalization: The Case of a Hydrophobic Coating”, 2017, Biomimetics, 2(4), 22 (doi:10.3390/biomimetics2040022);

### CLAUSES

For reasons of completeness, various aspects of the invention are set out in the following numbered clauses:

Clause 1. A process for preparing a catecholamine-based membrane, the process comprising the steps of:
a) cross-linking a catechol derivative with an amine selected from the group consisting of:
a.1) a known aliphatic amine hydrocarbon of formula (II)

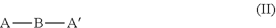

wherein
A and A′ are the same or different and represent —NR₁R′₁,
B represents a (C₁-C₂₀)alkylene; (C₁-C₂₀)alkylene substituted with one or more substituents selected from the group consisting of: —OH, halogen, —NO₂, cyano, —O—(C₁-C₁₀)alkyl, —C(O)OR₂, and —NR₃R′₃; (C₂-C₂₀)alkenylene; (C₂-C₂₀)alkenylene substituted with one or more substituents selected from the group consisting of: —OH, halogen, —NO₂, cyano, —O—(C₁-C₁₀)alkyl, —C(O)OR₄, and —NR₅R′₅; (C₂-C₂₀)alkynylene; (C₂-C₂₀)alkynylene substituted with one or more substituents selected from the group consisting of: —OH, halogen, —NO₂, cyano, —O—(C₁-C₁₀)alkyl, —C(O)OR₆, and —NR₇R′₇; a known 3- to 20-membered heteroalkylene; a known 3- to 20-membered heteroalkylene substituted with one or more substituents selected from the group consisting of: —OH, halogen, —NO₂, cyano, —O—(C₁-C₁₀)alkyl, —C(O)OR₈, and NR₉R′₉; a known 3- to 20-membered heteroalkenylene; and a known 3- to 20-membered heteroalkenylene substituted with one or more substituents selected from the group consisting of: —OH, halogen, —NO₂, cyano, —O—(C₁-C₁₀)alkyl, —C(O)OR₁₀, and —NR₁₁R′₁₁;
A is bound to the first atom member forming part of the B biradical hydrocarbon backbone;
A′ is bound to the last atom member forming part of the B biradical hydrocarbon backbone;
wherein the “first” and “last” atom members are identified reading the B biradical hydrocarbon backbone from left to right or vice versa;
R₁, R′₁, R₃, R′₃, R₅, R′s, R₇, R′₇, R₉, R′₉, R₁₁, and R′₁₁ are the same or different and are selected from the group consisting of: —H; (C₁-C₁₀)alkyl; (C₁-C₁₀)haloalkyl; (C₂-C₁₀)alkenyl; (C₂-C₁₀)alkynyl; and (C₁-C₁₀)alkyl substituted with one or more substituents selected from the group consisting of —OH, —NO₂, cyano, —O—(C₁-C₁₀)alkyl, —C(O)OR₁₂, and —NR₁₃R′₁₃;
R₂, R₄, R₆, R₈, R₁₀ and R₁₂ are independently selected from the group consisting of —H; (C₁-C₁₀)alkyl; (C₁-C₁₀)haloalkyl; (C₂-C₁₀)alkenyl; (C₂-C₁₀)alkynyl; and (C₁-C₁₀)alkyl substituted with one or more substituents selected from the group consisting of —OH, —NO₂, cyano, —O—(C₁-C₁₀)alkyl, —C(O)OR₁₄ and —NR₁₅R′₁₅;
R₁₃, R′₁₃, R₁₄, R₁₅ and R′₁₅ are the same or different and are selected from the group consisting of: —H; (C₁-C₁₀)alkyl; (C₁-C₁₀)haloalkyl; (C₂-C₁₀)alkenyl; and (C₂-C₁₀)alkynyl;
a “known 3- to 20-membered heteroalkylene” means a known saturated chain consisting of from 3 to 20 members selected from the group consisting of C(Rₓ)₂, CRₓ, —N—, —NR′ₓ—, —S—, and —O—, provided that: (a) at least one of the members is —N—, —NR′ₓ—, —S—, or —O—; and (b) the first and the last members forming the backbone of the heteroalkylene are carbon atoms;
a “known 3 to 20-membered heteroalkenylene” means a known unsaturated chain consisting of from 3 to 20 members selected from the group consisting of C(Rₓ)₂, —CRₓ—, —N—, —NR′ₓ—, —S—, and —O—, provided that: (a) at least one of the members is —N—, —NR′ₓ—, —S—, or —O—; (b) the first and the last members forming the backbone of the heteroalkylene are carbon atoms; and (c) includes one or more double bonds;
   Rₓ is independently selected from the group consisting of —H; —OH; (C₁-C₁₀)alkyl; (C₂-C₁₀)alkenyl; (C₂-C₁₀)alkynyl; (C₁-C₁₀)haloalkyl; O—(C₁-C₁₀)alkyl; —O—(C₂-C₁₀)alkenyl; —O—(C₂-C₁₀)alkynyl; nitro, —NRₓ₁Rₓ₂; (C₁-C₁₀)alkyl substituted with one or more substituents selected from the group consisting of —OH, —NO₂, cyano, —O—(C₁-C₁₀)alkyl, —C(O)OR₁₆, and —NR₁₇R′₁₇; and halogen; and
   R_{X1}, Rₓ₂, R₁₆, R₁₇, R′₁₇, and R′ₓ are independently selected from the group consisting of —H, (C₁-C₁₀)alkyl, (C₂-C₁₀)alkenyl, (C₂-C₁₀)alkynyl, and (C₁-C₁₀)haloalkyl; and
a.2) an aromatic amine of formula (IIbis):

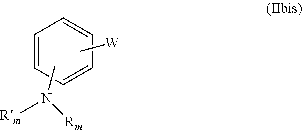

wherein W represents:
—NRₜR′ₜ or *S-S-L; wherein: (*) denotes that the S atom of W radical is bound to a carbon atom forming part of the aromatic ring; and
L is selected from the group consisting of: (C₁-C₁₀)alkyl substituted with one or more substituents selected from the group consisting of —OH, —NO₂, cyano, —O—(C₁-C₁₀)alkyl, —C(O)OR₂₈, and —NR₂₈R′₂₈, provided that at least one of the substituents is —NR₂₈R′₂₈; (C₁-C₁₀)haloalkyl substituted with one or more substituents selected from the group consisting of —OH, —NO₂, cyano, —O—(C₁-C₁₀)alkyl, —C(O)OR₂₈, and —NR₂₈R′₂₈, provided that at least one of the substituents is —NR₂₈R′₂₈; (C₂-C₁₀)alkenyl substituted with one or more substituents selected from the group consisting of —OH, —NO₂, cyano, —O—(C₁-C₁₀)alkyl, —C(O)OR₂₈, and —NR₂₈R′₂₈, provided that at least one of the substituents is —NR₂₈R′₂₈; (C₂-C₁₀)alkynyl substituted with one or more substituents selected from the group consisting of —OH, —NO₂, cyano, —O—(C₁-C₁₀)alkyl, —C(O)OR₂₈, and —NR₂₈R′₂₈, provided that at least one of the substituents is —NR₂₈R′₂₈; and a known aromatic ring having 5 or 6 members selected from the group consisting of —CR_{z}—, —N—, —O—, —NR′ᵥ, —S—;
Rₘ, R′ₘ, Rₜ and R′ₜ are the same or different and are selected from the group consisting of: H, (C₁-C₁₀)alkyl; (C₁-C₁₀)haloalkyl; (C₂-C₁₀)alkenyl; (C₂-C₁₀)alkynyl; (C₁-C₁₀)alkyl substituted with one or more substituents selected from the group consisting of —OH, —NO₂, cyano, —O—(C₁-C₁₀)alkyl, —C(O)OR₂₉, and —NR₃₀R′₃₀; and a known aromatic ring having 5 or 6 members selected from the group consisting of —CR_{z}—, —N—, —NR′_{z}, —O—, and —S—;
at least one of R_{y}, is —NR₃₁R′₃₁ and the other(s) Rᵥ are selected from the group consisting of H, —NR₃₁R′₃₁, (C₁-C₁₀)alkyl; (C₁-C₁₀)haloalkyl; (C₂-C₁₀)alkenyl; (C₂-C₁₀)alkynyl; and (C₁-C₁₀)alkyl substituted with one or more substituents selected from the group consisting of —OH, —NO₂, cyano, —O—(C₁-C₁₀)alkyl, —C(O)OR₃₂, and —NR₃₃R′₃₃;
R_{z}, and R′_{z} are independently selected from the group consisting of: H, (C₁-C₁₀)alkyl; (C₁-C₁₀)haloalkyl; (C₂-C₁₀)alkenyl; (C₂-C₁₀)alkynyl; (C₁-C₁₀)alkyl substituted with one or more substituents selected from the group consisting of —OH, —NO₂, cyano, —O—(C₁-C₁₀)alkyl, —C(O)OR₃₄, and —NR₃₅R′₃₅; and

R₂₈, R′₂₈, R₂₉, R₃₀, R′₃₀, R₃₁, R′₃₁, R₃₂, R₃₃, R′₃₃, R₃₄, R₃₅, and R′₃₅ are independently selected from the group consisting of: —H; (C₁-C₁₀)alkyl; (C₁-C₁₀)haloalkyl; (C₂-C₁₀)alkenyl; and (C₂-C₁₀)alkynyl;
to create a catecholamine membrane in the air/liquid interface in the absence of any support, the crosslinking reaction being performed in a liquid medium, wherein: both the catechol and the amine are soluble, and the pH is from 6.5 to 10, particularly from 6.5 to 8; and under agitation, particularly appropriate agitation; and
b) isolating the membrane resulting from step (a) from the air/liquid interface.

Clause 2. The process of clause 1, wherein the amine is an amine of formula (II).

Clause 3. The process of any of the preceding clauses, wherein A and A′ are the same or different and represent NHR′.

Clause 4. The process of any one of the preceding clauses, wherein the known aliphatic amine of formula (II) is one wherein A and A′ are the same.

Clause 5. The process of any of the preceding clauses, wherein A and A′ are the same and represent NHR′₁.

Clause 6. The process of any of the preceding clauses, wherein at least one of A and A′ represents —NH₂.

Clause 7. The process of any of the preceding clauses, wherein A and A′ are the same and represent —NHR′₁, particularly —NH₂.

Clause 8. The process of any of the preceding clauses, wherein B represents: (C₁-C₂₀)alkylene; (C₁-C₂₀)alkylene substituted as defined in clause 1; or a known 3- to 20-membered heteroalkylene as defined in clause 1.

Clause 9. The process of any of the preceding clauses, wherein B represents (C₁-C₁₅)alkylene; (C₁-C₁₅)alkylene substituted as defined in clause 1; or a known 3- to 15-membered heteroalkylene as defined in clause 1.

Clause 10. The process of any of the preceding clauses, wherein B represents (C₁-C₁₅)alkylene, (C₂-C₁₅)alkylene, (C₄-C₁₅)alkylene or (C₅-C₁₅)alkylene.

Clause 11. The process of any of the preceding clauses, wherein B represents a known 3- to 15-membered heteroalkylene, particularly represents a known 3- to 10-membered heteroalkylene or a known 4- to 8-membered heteroalkylene.

Clause 12. The process of any of the preceding clauses, wherein B represents a known 3- to 15-membered heteroalkylene, particularly represents a 3- to 10-membered heteroalkylene, wherein each one of the members is selected from the group consisting of: C(Rₓ)₂, CRₓ, —NR′ₓ—, and —S—, wherein Rₓ and R′ₓ are as defined in clause 1.

Clause 13. The process of any of the preceding clauses, wherein B represents a known 4- to 15-membered heteroalkylene, particularly represents a known 4- to 10-membered heteroalkylene, wherein each one of the members is selected from the group consisting of C(Rₓ)₂, CRₓ, —NR′ₓ—, and —S—, wherein Rₓ and R′ₓ are as defined in clause 1.

Clause 14. The process of any of the preceding clauses, wherein B represents a known 3- to 15-membered heteroalkylene, particularly represents a known 3- to 10-membered heteroalkylene, wherein:
(a) one or two of the members are —S— atoms and the others are carbon atoms selected from C(Rₓ)₂, and CRₓ; or, alternatively,
(b) one or two of the members are —NR′ₓ—, and the others are carbon atoms selected from the group consisting of C(Rₓ)₂, and CRₓ; wherein Rₓ is as defined in clause 1;
(c) one of the members is —S— atoms and the others are carbon atoms selected from the group consisting of C(Rₓ)₂, and CRₓ; or, alternatively,
(d) one of the members is —NR′ₓ—, and the others are carbon atoms selected from the group consisting of C(Rₓ)₂, and CRₓ;
(e) two of the members are —S— atoms and the others are carbon atoms selected from the group consisting of C(Rₓ)₂, and CRₓ; or, alternatively,
(f) two of the members are NR′ₓ—, and the others are carbon atoms selected from the group consisting of C(Rₓ)₂, and CRₓ;
wherein Rₓ and R′ₓ are as defined in clause 1.

Clause 15. The process according to any one of the previous clauses, wherein the known aliphatic amine of formula (II) is selected from the group consisting of an amine of formula (II1), an amino of formula (II2), and an amine of formula (II3):

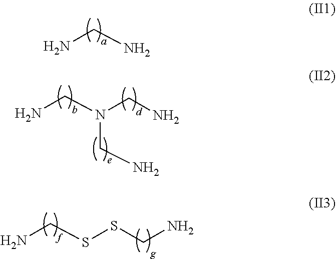

wherein a is an integer from 1 to 20; b, d, and e are independently an integer from 1 to 10, more particularly b, d, and e are the same; and f, and g are independently an integer from 1 to 10, more particularly f and g are the same; more particularly is selected from the group consisting of: hexamethylenediamine, octamethylenediamine, dodecamethylenediamine, cystamine, tris-(3-aminopropyl)amine, and tris-(2-aminopropyl)amine.

Clause 16. The process according to clause 1, wherein the amine is an aromatic amine of formula (IIbis).

Clause 17. The process according to the previous clause, wherein the amine is an aromatic amine of formula (IIbis1):

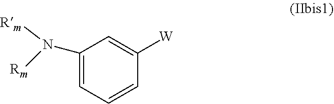

Wherein Rₘ, R′ₘ, and W are as defined in clause 1.

Clause 18. The process according to any of the clauses 1, 16-17, wherein the amine is an aromatic amine of formula (IIbis) or (IIbis1), and W represents —NRₜR′ₜ.

Clause 19. The process according to any of the clauses 1, 16-18, wherein the amine is an aromatic amine of formula (IIbis) or (IIbis1), W represents —NRₜR′ₜ, and Rₘ, R′ₘ, Rₜ and R′ₜ are the same.

Clause 20. The process according to clause 19, wherein Rₘ, R′ₘ, Rₜ and R′ₜ are the same and represent —H.

Clause 21. The process according to any of the preceding clauses, wherein Rₜ and R′ₜ are the same and are other than hydrogen.

Clause 22. The process according to any of the preceding clauses, wherein Rₘ and R′ₘ are hydrogen; and Rₜ and R′ₜ are the same and are other than hydrogen.

Clause 23. The process according to any of the preceding clauses, wherein each one of Rₜ and R′ₜ represent an aromatic ring as defined in clause 1.

Clause 24. The process according to any of the preceding clauses, each one of Rₜ and R′ₜ represent an aromatic ring having 6 members as defined in clause 1.

Clause 25. The process according to any of the preceding clauses, wherein each one of Rₜ and R′ₜ represent an aromatic ring having 6 members, all the members being —CR_{z}—, wherein R_{z} is as defined in clause 1.

Clause 26. The process according to any of the preceding clauses, wherein Rₜ and R′ₜ are the same and represent an aromatic ring having 6 members, all the members, being —CR_{z}— members, wherein R_{z} is as defined in clause 1.

Clause 27. The process according to any of the preceding clauses, wherein the amine is one of formula (IIbis) or (IIbis1), being W═NRₜR′ₜ, and one or more of Rₘ, R′ₘ, Rₜ and R′ₜ having the following formula (IV):

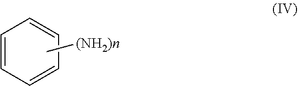

wherein n is 1 or 2, particularly 1, and the other(s) being —H.

Clause 28. The process according to the preceding clause, wherein the amine of formula (IIbis) is one of formula (V) or (VI):

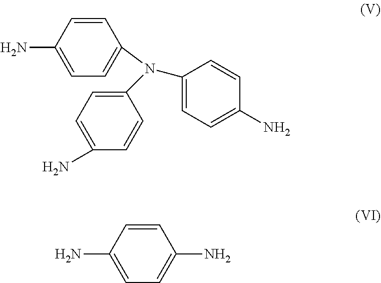 [SMILES: Nc1ccc(N(c2ccc(N)cc2)c2ccc(N)cc2)cc1 ; Nc1ccc(N)cc1]

Clause 29. The process according to any of the preceding clauses, wherein the amine is an aromatic amine of formula (IIbis) or (IIbis1), and W represents *S-S-L.

Clause 30. The process according to any of the preceding clauses, wherein the amine is an aromatic amine of formula (IIbis) or (IIbis1), W represents *S-S-L, and Rₘ and R′ₘ are the same.

Clause 31. The process according to any of the preceding clauses, wherein the amine is an aromatic amine of formula (IIbis) or (IIbis1), W represents *S-S-L, and Rₘ and R′ₘ represent —H.

Clause 32. The process according to any of the preceding clauses, wherein the amine is an aromatic amine of formula (IIbis) or (IIbis1), L represents an aromatic ring as defined in clause 1.

Clause 33. The process according to any of the preceding clauses, wherein L represents an aromatic ring having 6 members as defined in clause 1.

Clause 34. The process according to any of the preceding clauses, wherein L represents an aromatic ring having 6 members, the same or different, represented by —CRᵥ—, as defined in clause 1.

Clause 35. The process according to any of the preceding clauses, wherein L has the following formula (IV):

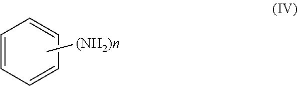

n 1 or 2, and
one of the carbon atoms forming the aromatic ring being bound to the —S— atom.

Clause 36. The process according to any of the preceding clauses, wherein the amine of formula (IIbis) corresponds to the amine of formula (VII):

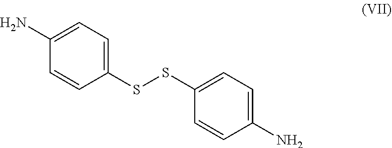 [SMILES: Nc1ccc(SSc2ccc(N)cc2)cc1]

Clause 37. The process according to any of the preceding clauses, wherein the amine is selected from the group consisting of: hexamethylenediamine, octamethylenediamine, dodecamethylenediamine, cystamine, tris-(3-aminopropyl)amine, tris-(2-aminopropyl)amine, and 4,4′,4″-triaminotriphenylamine.

Clause 38. The process according to any one of the previous clauses, wherein the catechol derivative is one of formula (III):

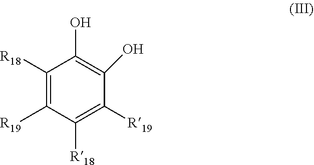

wherein
R₁₈, R₁₉, R′₁₈ and R′₁₉ are the same or different and are selected from the group consisting of:
   —H;
   —OH;
   —NR₂₀R′₂₀;
   halogen
   (C₁-C₁₀)alkyl;
   (C₂-C₁₀)alkenyl;
   (C₁-C₁₀)alkyl substituted with one or more substituents selected from the group consisting of: —OH, halogen, nitro, cyano, (C₁-C₁₀)alkyl, (C₁-C₁₀)haloalkyl, —NR₂₁R′₂₁, —C(O)OR₂₂, and —O—(C₁-C₁₀)alkyl;
   (C₂-C₁₀)alkenyl substituted with one or more substituents selected from the group consisting of: —OH, halogen, nitro, cyano, (C₁-C₁₀)alkyl, (C₁-C₁₀)haloalkyl, —NR₂₈R′₂₃, —C(O)OR₂₄, and —O—(C₁-C₁₀)alkyl;
   a known ring system consisting of one or two rings, each one of the rings: (a) consisting of 5 or 6 members selected from the group consisting of —C(R_{y})₂—, —CR_{y}—, —N—, —NR′_{y}, —S—, and —O—; (b) being saturated, partially unsaturated or aromatic, and (c) being isolated, partially isolated or fused;
each one of the R_{y} is independently selected from the group consisting of —H, —OH, (C₁-C₁₀)alkyl, (C₂-C₁₀)alkenyl, (C₂-C₁₀)alkynyl, (C₁-C₁₀)haloalkyl, —O—(C₁-C₁₀)alkyl, nitro, —NR₂₅R₂₅, and halogen;
R′_{y} is selected from the group consisting of —H, (C₁-C₁₀)alkyl, (C₂-C₁₀)alkenyl, (C₂-C₁₀)alkynyl, and (C₁-C₁₀)haloalkyl;
R₂₀, R′₂₀, R₂₁, R′₂₁, R₂₃, R′₂₃, R₂₅, and R′₂₅ are the same or different and are selected from the group consisting of: —H; (C₁-C₁₀)alkyl; (C₁-C₁₀)haloalkyl; (C₂-C₁₀)alkenyl; (C₂-C₁₀)alkynyl; (C₁-C₁₀)alkyl substituted with one or more substituents selected from the group consisting of: —OH, halogen, nitro, cyano, (C₁-C₁₀)alkyl, (C₁-C₁₀)haloalkyl, —NR₂₆R′₂₆, —C(O)OR₂₇, and —O—(C₁-C₁₀)alkyl;
R₂₂ and R₂₄ are independently selected from the group consisting of: H, (C₁-C₁₀)alkyl, (C₁-C₁₀)haloalkyl, (C₂-C₁₀)alkenyl; and (C₂-C₁₀)alkynyl; and
R₂₆, R′₂₆ and R₂₇ are the same or different and are selected from the group consisting of: —H; (C₁-C₁₀)alkyl; (C₁-C₁₀)haloalkyl; (C₂-C₁₀)alkenyl; and (C₂-C₁₀)alkynyl;

Clause 39. The process of any one of the preceding clauses, wherein the catechol derivative is one of formula (III) wherein:
R₁₈ and R₁₉ are the same or different and are selected from the group consisting of: —H; —OH; (C₁-C₁₀)alkyl; (C₁-C₁₀)alkyl substituted as defined in clause 38; and (C₂-C₁₀)alkenyl substituted as defined in clause 38; or, alternatively,
one of R₁₈ and R₁₉ is a known ring system consisting of two rings, each one of the rings (a) consisting of 5-6 members selected from the group consisting of —C(R_{y})₂—, —CR_{y}—, —N—, —NR′_{y}—, —S—, and —O—, (b) being saturated, partially unsaturated or aromatic, and (c) being isolated, partially isolated or fused; wherein R_{y} and R′_{y} are as defined in clause 38; or, alternatively,
one of R₁₈ and R₁₉ is a known ring system consisting of two rings, each one of the rings (a) consisting of 6 members selected from the group consisting of —C(R_{y})₂—, —CR_{y}—, —N—, —NR′_{y}—, —S—, and —O—, (b) being saturated, partially unsaturated or aromatic, and (c) being fused; wherein R_{y} and R′_{y} are as defined in clause 38; or, alternatively,
one of R₁₈ and R₁₉ is a known ring system consisting of two rings, each one of the rings (a) consisting of 6 members selected from the group consisting of —C(R_{y})₂—, —CR_{y}—, —N—, —NR′_{y}—, —S—, and —O—, provided that at least one of the rings includes a heteroatom (—N—, —NR′_{y}, —S—, and —O—); (b) being saturated, partially unsaturated or aromatic, and (c) being fused; wherein R_{y} and R′_{y} are as defined in clause 38; or, alternatively,
one of R₁₈ and R₁₉ is a known ring system consisting of two rings, each one of the rings (a) consisting of 6 members selected from the group consisting of —C(R_{y})₂—, —CR_{y}—, —N—, —NR′_{y}—, —S—, and —O—, provided that at least one of the rings includes a —O— heteroatom; (b) being saturated, partially unsaturated or aromatic, and (c) being fused; wherein R_{y} and R′_{y} are as defined in clause 38; or, alternatively,
one of R₁₈ and R₁₉ is a known ring system consisting of two rings, each one of the rings (a) consisting of 6 members selected from the group consisting of —C(R_{y})₂—, —CR_{y}—, —O—, provided that at least one of the rings includes a —O-heteroatom; (b) being saturated, partially unsaturated or aromatic, and (c) being fused; wherein R_{y} and R′_{y} are as defined in clause 38; or, alternatively,
one of R₁₈ and R₁₉ is a known ring system consisting of two rings, each one of the rings (a) consisting of 6 members selected from the group consisting of —C(R_{y})₂—, —CR_{y}—, —O—, provided that only one of the rings includes a —O-heteroatom; (b) being saturated, partially unsaturated or aromatic, and (c) being fused; wherein R_{y} and R′_{y} are as defined in clause 38.

Clause 40. The process according to any of the preceding clauses, wherein the catechol derivative is one of formula (III) wherein one of the R₁₈ or R₁₉ is —H and the other is as defined in any of the above clauses, in particular in clauses 38-39.

Clause 41. The process according to any of the preceding clauses, wherein the catechol derivative of formula (III) is one wherein R′1₈ and R′₁i are —H, and R₁₈ and R₁₉ are as defined in any of the previous clauses 39-40 or 38.

Clause 42. The process according to any of the preceding clauses, wherein the catechol derivative is selected from the group consisting of: pyrocatechol, caffeic acid, dopamine, 4-methylcatechol, pyrogallol, and catechin.

Clause 43. The process according to any one of the previous clauses, wherein the pH is comprised from 6.5 to 8 or from 7 to 7.5; or, the pH is selected from the group consisting of 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, 9, 9.1, 9.2, 9.3, 9.4, 9.5, 9.6, 9.7, 9.8, 9.9, and 10.

Clause 44. The process according to any one of the previous clauses, wherein
a) agitation is performed during the whole step of cross-linking or alternatively during part of the cross-linking reaction, or alternatively
b) agitation is performed during the whole step of cross-linking such that no turbulences are provided, more particularly at a stirring speed is equal to or below than 450 rpm, equal to or below than 400 rpm, or equal to below than 350 rpm, or alternatively
c) agitation is performed using the lowest speed possible to avoid the turbulence in the air/liquid interface, being homogeneous during the whole procedure and avoiding erratic movement of the magnet or mechanical pieces involved in the agitation procedure, more particularly at a stirring speed is equal to or below than 450 rpm, equal to or below than 400 rpm, or equal to below than 350 rpm, or alternatively
d) agitation is only performed during part of the cross-linking reaction, in particular agitation is performed such that no turbulences are provided until a change in coloration is observed and then it is stopped, even more particularly agitation is performed for a period of time from 10 min to 2 hours and then it is stopped. or alternatively
e) the agitation speed is equal to or below than 400 rpm, or equal to or below than 350 rpm.

Clause 45. The process according to any one of the previous clauses, wherein the amine of formula (II) or (IIbis) is at a molar ratio excess with respect to the catechol derivative.

Clause 46. The process according to any one of the previous clauses, wherein the crosslinking step is performed at a temperature comprised from 10 to 60° C.

Clause 47. The process according to any one of the previous clauses, wherein the crosslinking step is performed for at least a period of 24 h.

Clause 48. The process according to any one of the previous clauses, wherein the liquid medium comprises an aqueous-based buffer, more particularly, an aqueous-based buffer in the absence of nitrogen atoms or amino groups, even more particularly, the aqueous-based buffer being selected from the group consisting of a phosphate buffer, a carbonate buffer and a citrate buffer.

Clause 49. The process according to any one of the previous clauses, wherein the liquid medium is water.

Clause 50. A self-standing catecholamine-based membrane obtainable by the process as defined in any one of the previous clauses.

Clause 51. The catecholamine-based membrane according to the preceding clause, which further comprises one or more molecules of interest selected from the group consisting of therapeutic molecules, cells, growth factors, detection labels (fluorescent active moieties, nanoparticles or antibodies), and combinations thereof.

Clause 52. The catecholamine-based membrane according to clause 51, wherein the molecule of interest is covalently bound to the membrane, particularly by an amide bond.

Clause 53. A process for preparing the catecholamine-based membranes as defined in the preceding two clauses, the process comprising:
(i.1) performing step (a) of the process as defined in clause 1,
(i.2) adding to the reaction medium the molecule of interest; and
(i.3) performing step (b) of the process as defined in clause 1;
or, alternatively, the process comprising:
(ii.1) incubating the catecholamine-based membrane as defined in clause 50 with the molecule of interest.

Clause 54. Use of the catecholamine-based membrane according to clause 50 as adhesive.

Clause 55. Use of the catecholamine-based membrane according to clause 50 as a vehicle of a molecule of interest such as a therapeutic or label molecule.

Clause 56. A catecholamine-based membrane as defined in any of the clauses 51 to 52, wherein the molecules of interest are therapeutic molecules for use in therapy, or alternatively a self-standing catecholamine-based membrane as defined in any of the clauses 47 to 48, wherein the molecules of interest are detection labels for use or diagnosis.

Clause 57. A catecholamine-based membrane as defined in any of the clauses 51 to 52, wherein the molecules of interest are cells, growth factors or combinations thereof for use in regenerating tissues.

Clause 58. An article partially or completely coated with the membrane as defined in any one of the clauses 50 to 52.

1. A process for preparing a self-standing catecholamine-based membrane, the process comprising the steps of:
a) crosslinking a catechol derivative with an amine selected from the group consisting of:
a.1) a known aliphatic amine hydrocarbon of formula (II) 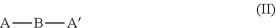
wherein
A and A′ are the same or different and are —NR₁R′₁,
B is selected from the group consisting of a (C₁-C₂₀)alkylene; (C₁-C₂₀)alkylene substituted with one or more substituents selected from the group consisting of: —OH, halogen, —NO₂, cyano, —O—(C₁-C₁₀)alkyl, —C(O)OR₂, and —NR₃R′₃; (C₂-C₂₀)alkenylene; (C₂-C₂₀)alkenylene substituted with one or more substituents selected from the group consisting of: —OH, halogen, —NO₂, cyano, —O—(C₁-C₁₀)alkyl, —C(O)OR₄, and —NR₅R′₅; (C₂-C₂₀)alkynylene; (C₂-C₂₀)alkynylene substituted with one or more substituents selected from the group consisting of: —OH, halogen, —NO₂, cyano, —O—(C₁-C₁₀)alkyl, —C(O)OR₆, and —NR₇R′₇; a known 3- to 20-membered heteroalkylene; a known 3- to 20-membered heteroalkylene substituted with one or more substituents selected from the group consisting of: —OH, halogen, —NO₂, cyano, —O—(C₁-C₁₀)alkyl, —C(O)OR₈, and NR₉R′₉; a known 3- to 20-membered heteroalkenylene; and a known 3- to 20-membered heteroalkenylene substituted with one or more substituents selected from the group consisting of: —OH, halogen, —NO₂, cyano, —O—(C₁-C₁₀)alkyl, —C(O)OR₁₀, and —NR₁₁R′₁₁;
A is bound to the first atom member forming part of the B biradical hydrocarbon backbone;
A′ is bound to the last atom member forming part of the B biradical hydrocarbon backbone;
wherein the “first” and “last” atom members are identified reading the B biradical hydrocarbon backbone from left to right or vice versa;
R₁, R′₁, R₃, R′₃, R₅, R′₅, R₇, R′₇, R₉, R′₉, R₁₁, and R′₁₁ are the same or different and are selected from the group consisting of: —H; (C₁-C₁₀)alkyl; (C₁-C₁₀)haloalkyl; (C₂-C₁₀)alkenyl; (C₂-C₁₀)alkynyl; (C₁-C₁₀)alkyl substituted with one or more substituents selected from the group consisting of —OH, —NO₂, cyano, —O—(C₁-C₁₀)alkyl, —C(O)OR₁₂, and —NR₁₃R′₁₃;
R₂, R₄, R₆, R₈, R₁₀ and R₁₂ are independently selected from the group consisting of —H; (C₁-C₁₀)alkyl; (C₁-C₁₀)haloalkyl; (C₂-C₁₀)alkenyl; (C₂-C₁₀)alkynyl; (C₁-C₁₀)alkyl substituted with one or more substituents selected from the group consisting of —OH, —NO₂, cyano, —O—(C₁-C₁₀)alkyl, —C(O)OR₁₄ and —NR₁₅R′₁₅;
R₁₃, R′₁₃, R₁₄, R₁₅ and R′₁₅ are the same or different and are selected from the group consisting of: —H; (C₁-C₁₀)alkyl; (C₁-C₁₀)haloalkyl; (C₂-C₁₀)alkenyl; and (C₂-C₁₀)alkynyl;
a “known 3- to 20-membered heteroalkylene” means a known saturated chain consisting of from 3 to 20 members selected from the group consisting of C(Rₓ)₂, CRₓ, —N—, —NR′ₓ—, —S—, and —O—, provided that: (a) at least one of the members is —N—, —NRₓ—, —S—, or —O—; and (b) the first and the last members forming the backbone of the heteroalkylene are carbon atoms;
a “known 3 to 20-membered heteroalkenylene” means a known unsaturated chain consisting of from 3 to 20 members selected from the group consisting of C(Rₓ)₂, —CRₓ—, —N—, —NR′ₓ—, —S—, and —O—, provided that: (a) at least one of the members is —N—, —NRₓ—, —S—, or —O—; (b) the first and the last members forming the backbone of the heteroalkylene are carbon atoms; and (c) includes one or more double bonds;
Rₓ is independently selected from the group consisting of —H; —OH; (C₁-C₁₀)alkyl; (C₂-C₁₀)alkenyl; (C₂-C₁₀)alkynyl; (C₁-C₁₀)haloalkyl; O—(C₁-C₁₀)alkyl; —O—(C₂-C₁₀)alkenyl; —O—(C₂-C₁₀)alkynyl; nitro, —NRₓ₁Rₓ₂; (C₁-C₁₀)alkyl substituted with one or more substituents selected from the group consisting of —OH, —NO₂, cyano, —O—(C₁-C₁₀)alkyl, —C(O)OR₁₆, and —NR₁₇R′₁₇; and halogen; and
R_{X1}, Rₓ₂, R₁₆, R₁₇, R′₁₇, and R′ₓ are independently selected from the group consisting of —H, (C₁-C₁₀)alkyl, (C₂-C₁₀)alkenyl, (C₂-C₁₀)alkynyl, and (C₁-C₁₀)haloalkyl; and
a.2) an aromatic amine of formula (IIbis): 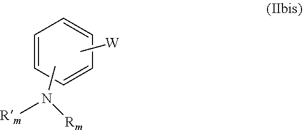
wherein W is:
—NRₜR′ₜ or
*S-S-L; wherein (*) denotes that the S atom of W radical is bound to a carbon atom forming part of the aromatic ring; and
L is selected from the group consisting of: (C₁-C₁₀)alkyl substituted with one or more substituents selected from the group consisting of —OH, —NO₂, cyano, —O—(C₁-C₁₀)alkyl, —C(O)OR₂₈, and —NR₂₈R′₂₈, provided that at least one of the substituents is —NR₂₈R′₂₈; (C₁-C₁₀)haloalkyl substituted with one or more substituents selected from the group consisting of —OH, —NO₂, cyano, —O—(C₁-C₁₀)alkyl, —C(O)OR₂₈, and —NR₂₈R′₂₈, provided that at least one of the substituents is —NR₂₈R′₂₈; (C₂-C₁₀)alkenyl substituted with one or more substituents selected from the group consisting of —OH, —NO₂, cyano, —O—(C₁-C₁₀)alkyl, —C(O)OR₂₈, and —NR₂₈R′₂₈, provided that at least one of the substituents is —NR₂₈R′₂₈; (C₂-C₁₀)alkynyl substituted with one or more substituents selected from the group consisting of —OH, —NO₂, cyano, —O—(C₁-C₁₀)alkyl, —C(O)OR₂₈, and —NR₂₈R′₂₈, provided that at least one of the substituents is —NR₂₈R′₂₈; and a known aromatic ring having 5 or 6 members selected from the group consisting of —CRᵥ—, —N—, —O—, —NR′ᵥ, and —S—;
Rₘ, R′ₘ, Rₜ and R′ₜ are the same or different and are selected from the group consisting of: H, (C₁-C₁₀)alkyl; (C₁-C₁₀)haloalkyl; (C₂-C₁₀)alkenyl; (C₂-C₁₀)alkynyl; (C₁-C₁₀)alkyl substituted with one or more substituents selected from the group consisting of —OH, —NO₂, cyano, —O—(C₁-C₁₀)alkyl, —C(O)OR₂₉, and —NR₃₀R′₃₀; and a known aromatic ring having 5 or 6 members selected from the group consisting of —CR_{z}—, —N—, —NR′_{z}, —O—, and —S—;
at least one of Rᵥ, is —NR₃₁R′₃₁ and the other(s)Rᵥ are selected from the group consisting of H, —NR₃₁R′₃₁, (C₁-C₁₀)alkyl; (C₁-C₁₀)haloalkyl; (C₂-C₁₀)alkenyl; (C₂-C₁₀)alkynyl; and (C₁-C₁₀)alkyl substituted with one or more substituents selected from the group consisting of —OH, —NO₂, cyano, —O—(C₁-C₁₀)alkyl, —C(O)OR₃₂, and —NR₃₃R′₃₃;
R_{z}, and R′_{z} are independently selected from the group consisting of: H, (C₁-C₁₀)alkyl; (C₁-C₁₀)haloalkyl; (C₂-C₁₀)alkenyl; (C₂-C₁₀)alkynyl; (C₁-C₁₀)alkyl substituted with one or more substituents selected from the group consisting of —OH, —NO₂, cyano, —O—(C₁-C₁₀)alkyl, —C(O)OR₃₄, and —NR₃₅R′₃₅; and
R₂₈, R′₂₈, R₂₉, R₃₀, R′₃₀, R₃₁, R′₃₁, R₃₂, R₃₃, R′₃₃, R₃₄, R₃₅, and R′₃₅ are independently selected from the group consisting of: —H; (C₁-C₁₀)alkyl; (C₁-C₁₀)haloalkyl; (C₂-C₁₀)alkenyl; and (C₂-C₁₀)alkynyl;
to create a catecholamine membrane in the air/liquid interface in the absence of any support, the crosslinking reaction being performed in a liquid medium, wherein both the catechol and the amine are soluble, at a pH comprised from 6.5 to 10, and under agitation; and
b) isolating the membrane resulting from step (a) from the air/liquid interface.

2. The process according to claim 1, wherein the amine is one of formula (II).

3. The process according tom claim 1, wherein A and A′ are the same and are —NHR′₁.

4. The process according to claim 1, wherein B is (C₁-C₁₅)alkylene; (C₁-C₁₅)alkylene substituted with one or more substituents selected from the group consisting of: —OH, halogen, —NO₂, cyano, —O—(C₁-C₁₀)alkyl, —C(O)OR₄, and —NR₅R′₅; or a known 3- to 15-membered heteroalkylene as defined in claim 1.

5. The process according to claim 1, wherein the amine is selected from the group consisting of: hexamethylenediamine, octamethylenediamine, dodecamethylenediamine, cystamine, tris-(3-aminopropyl)amine, tris-(2-aminopropyl)amine, and 4,4′,4″-triaminotriphenylamine.

6. The process according to claim 1, wherein the catechol derivative is one of formula (III): 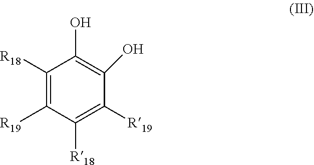
wherein
R₁₈, R₁₉, R′₁₈ and R′₁₉ are the same or different and are selected from the group consisting of:
—H;
—OH;
—NR₂₀R′₂₀;
halogen,
(C₁-C₁₀)alkyl;
(C₂-C₁₀)alkenyl;
(C₁-C₁₀)alkyl substituted with one or more substituents selected from the group consisting of: —OH, halogen, nitro, cyano, (C₁-C₁₀)alkyl, (C₁-C₁₀)haloalkyl, —NR₂₁R′₂₁, —C(O)OR₂₂, and —O—(C₁-C₁₀)alkyl;
(C₂-C₁₀)alkenyl substituted with one or more substituents selected from the group consisting of: —OH, halogen, nitro, cyano, (C₁-C₁₀)alkyl, (C₁-C₁₀)haloalkyl, —NR₂₈R′₂₃, —C(O)OR₂₄, and —O—(C₁-C₁₀)alkyl; and
a known ring system consisting of one or two rings, each one of the rings: (a) consisting of 5 or 6 members selected from the group consisting of —C(R_{y})₂—, —CR_{y}—, —N—, —NR′_{y}, —S—, and —O—; (b) being saturated, partially unsaturated or aromatic, and (c) being isolated, partially isolated or fused;
each one of the R_{y} is independently selected from the group consisting of —H, —OH, (C₁-C₁₀)alkyl, (C₂-C₁₀)alkenyl, (C₂-C₁₀)alkynyl, (C₁-C₁₀)haloalkyl, —O—(C₁-C₁₀)alkyl, nitro, —NR₂₅R₂₅, and halogen;
R′_{y} is selected from the group consisting of —H, (C₁-C₁₀)alkyl, (C₂-C₁₀)alkenyl, (C₂-C₁₀)alkynyl, and (C₁-C₁₀)haloalkyl;
R₂₀, R′₂₀, R₂₁, R′₂₁, R₂₃, R′₂₃, R₂₅, and R′₂₅ are the same or different and are selected from the group consisting of: —H; (C₁-C₁₀)alkyl; (C₁-C₁₀)haloalkyl; (C₂-C₁₀)alkenyl; (C₂-C₁₀)alkynyl; (C₁-C₁₀)alkyl substituted with one or more substituents selected from the group consisting of: —OH, halogen, nitro, cyano, (C₁-C₁₀)alkyl, (C₁-C₁₀)haloalkyl, —NR₂₆R′₂₆, —C(O)OR₂₇, and —O—(C₁-C₁₀)alkyl;
R₂₂ and R₂₄ are independently selected from the group consisting of: H, (C₁-C₁₀)alkyl, (C₁-C₁₀)haloalkyl, (C₂-C₁₀)alkenyl; and (C₂-C₁₀)alkynyl; and
R₂₆, R′₂₆ and R₂₇ are the same or different and are selected from the group consisting of: —H; (C₁-C₁₀)alkyl; (C₁-C₁₀)haloalkyl; (C₂-C₁₀)alkenyl; and (C₂-C₁₀)alkynyl.

7. The process according to claim 1, wherein the catechol derivative is selected from the group consisting of pyrocatechol, dopamine, pyrogallol, caffeic acid, 4-methylcatechol, and catechin.

8. The process according to claim 1, wherein the pH is from 6.5 to 8.

9. The process according to claim 1, wherein the known aliphatic amine hydrocarbon of formula (II) is at a molar ratio excess with respect to the catechol derivative.

10. A self-standing catecholamine-based membrane produced by the process as defined in claim 1.

11. The self-standing catecholamine-based membrane according to claim 10, which further comprises one or more molecules of interest selected from therapeutic molecules and detection labels.

12. A method of using the catecholamine-based membrane according to claim 10 as an adhesive, the method comprising
coating an article.

13. A method of using the catecholamine-based membrane according to claim 10 as a vehicle of a molecule of interests.

14. (canceled)

15. An article partially or completely coated with the membrane as defined in claim 10.

16. The process according to claim 1, wherein A and A′ are —NH₂.

17. A self-standing catecholamine-based membrane produced by the process as defined in claim 2.

18. A self-standing catecholamine-based membrane produced by the process as defined in claim 3.

19. A self-standing catecholamine-based membrane produced by the process as defined in claim 4.

20. A self-standing catecholamine-based membrane produced by the process as defined in claim 5.

21. A self-standing catecholamine-based membrane produced by the process as defined in claim 6.
